(19) United States
(12) Patent Application Publication
(10) Pub. No.: US 2026/0070873 A1
(43) Pub. Date: Mar. 12, 2026

(54) CORDYLUTENES WITH EXTRAORDINARY EFFECT IN BLUE LIGHT ABSORPTION

(71) Applicant: JOINCARING CO., LTD., Taipei (TW)

(72) Inventors: Po-Wei YU, Taipei City (TW); Ya-Lin CHANG, Taipei City (TW); Schung-Ching CHIU, Taipei City (TW)

(73) Assignee: JOINCARING CO., LTD., Taipei (TW)

(21) Appl. No.: 19/327,269

(22) Filed: Sep. 12, 2025

Related U.S. Application Data
(60) Provisional application No. 63/693,794, filed on Sep. 12, 2024.

(51) Int. Cl. C07C 233/49; A61K 31/202; A61P 27/02

(52) U.S. Cl. CPC C07C 233/49; A61K 31/202; A61P 27/02

(57) ABSTRACT

The present invention pertains to new compounds, possessing unique structural features, effective in blue light absorption. The compounds, named as Cordylutenes, show significant biological activity in the prevention and/or treatment of blue light-induced ocular damage. Also provided includes a method and a pharmaceutical, cosmetical or edible composition for preventing or treating an ocular disease, such as dry eye disease, cataracts, glaucoma, or macular degeneration, comprising the Cordylutenes or a pharmaceutically, cosmetically, or edibly acceptable salt thereof.

### CROSS REFERENCE TO RELATED APPLICATIONS

This application claims the priority benefit of U.S. Provisional Application No. 63/693,794 filed Sep. 12, 2024, which is fully incorporated herein in its entirety.

### FIELD OF THE INVENTION

The present invention pertains to new compounds which have a characteristic of high water-solubility and an extraordinary effect in blue light absorption.

### BACKGROUND OF THE INVENTION

In an era dominated by digital screens, the impact of prolonged exposure to blue light on ocular health is a growing concern. This ubiquitous light source, emitted by electronic devices, raises questions about its consequences on the delicate structures of the eye. This narrative explores the intricate relationship between blue light exposure and ocular tissues, shedding light on the mechanisms of oxidative damage and inflammation. From the ocular surface to the crystalline lens and retina, each segment faces potential harm, with implications for conditions such as dry eye, cataracts, and age-related macular degeneration (AMD).

The ocular surface is the very first barrier between the visual system and the external environment. It shields the eye from exposure to light sources that emit significantly in the blue spectrum, yet it is susceptible to light hazards and abnormalities. Prolonged exposure of the eyes to excessive blue light has been proven to induce a range of changes [1]. Research has also revealed that the impact of blue light hazard on the ocular surface is associated with oxidative stress damage, inflammation of the ocular surface, and cell apoptosis.

The excessive production of reactive oxygen species (ROS) leads to oxidative stress has been associated with aging and various age-related chronic conditions, including several eye-related pathologies such as dry eye disease, cataracts, glaucoma, and AMD [2].

Age-related macular degeneration is a significant cause of vision impairment in the elderly. It is characterized by the deterioration of central vision resulting from damage to retinal pigment epithelium cells (RPE) cells and photoreceptors [4]. Numerous studies involving animal models or human retinal cells have documented significant photochemical damage to the retina, which dramatically increased the levels of both total cellular and mitochondrial ROS, proposing blue light as a risk factor for AMD progression [3][4][5].

Marek et al. investigated the phototoxic effects of blue light illumination on human epithelial cells of the ocular surface. Exposure to blue light led to a significant reduction in cellular viability, alterations in cellular morphology, and an excessive production of ROS. Furthermore, hyperosmolar stress intensified the phototoxic effects of blue light, resulting in increased inflammation, disruption of mitochondrial membrane potential, and activation of the glutathione-based antioxidant system [6].

In addition, an animal study showed that tear volume and the tear film break-up time (TBUT) significantly decreased in blue light-irradiated mice, as well as a significant increase in the corneal levels of IL-1β and IL-6 [7]. This indicates that prolonged exposure to blue light has the potential to elevate inflammatory markers and oxidative stress, consequently exacerbating clinical parameters of dry eye in mice.

Blue light and radiation, in general, known to harm the retina, may also have the potential to cause photodynamic damage in aging lenses [8]. The lens's absorption of blue light is facilitated by structural proteins, protein metabolites, enzymes that absorb blue light, and generate yellow pigments. This process, particularly through the production of ROS and oxygen singlet after irradiation exposure, gradually results in lens darkening and yellowing, then ultimately contributing to aging and the development of cataracts [9]. The retina, a vital component in visual processing, comprises photoreceptors (rod and cone cells) and RPE. Sustaining the normal functioning of both photoreceptors and RPE cells is essential for the development of vision. Extensive exposure to blue light has been demonstrated to induce significant photochemical damage to the retina. Increasing evidence indicates that blue light leads to a substantial rise in ROS production, contributing to strong damage to photoreceptors, lipid peroxidation, and the induction of necrosis [1][3]. Meanwhile, the synergistic impact of blue light, N-retinylidene-N-retinylethanolamine (A2E), retinal, and photo-reversal of bleaching exacerbates photochemical damage, triggering inflammatory reactions, DNA damage, and the inhibition of mitochondria and lysosome function.

There are a variety of antioxidant molecules, such as vitamin C, catechins, polyphenols, and anthocyanin are tasked with protecting and maintaining antioxidant homeostasis in the eye. Oxidative stress that instigates the functional changes of the eye is similarly found in ocular conditions associated with aging, such as dry eye, glaucoma, cataracts, and macular degeneration. The role of antioxidants in preventing oxidative stress-related ocular dysfunctions is paramount.

However, some eye-protective substances like lutein or zeaxanthin, which is lipid-soluble, show problems in water solubility. Therefore, it is necessary to develop new and better solution for prevention and treatment of blue light-induced ocular damages.

### SUMMARY OF THE INVENTION

It is unexpectedly discovered in the present invention that new compounds are synthesized, providing blue light absorption capabilities and better water solubility.

In one aspect, the present invention provides a compound, named as Cordylutene, which has the structure of general Formula (I) below, or a pharmaceutically, cosmetically or edibly acceptable salt thereof.

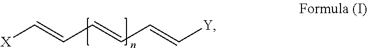

wherein
X and Y are the same or different, independently selected from the group consisting of aldehyde, ketone, carboxylic acid, ester, amide, acyl halide, acid anhydride, imide, and thioester; and
n is an integer from 5 to 9.

In one further aspect, the present invention provides a method for preventing or treating an ocular disease in a subject comprising administrating the subject a pharmaceutical, cosmetical or edible composition comprising a therapeutically effective amount of the Cordylutene, or a pharmaceutically, cosmetically or edibly acceptable salt thereof, or a mixture thereof.

In one yet aspect, the present invention provides a pharmaceutical, cosmetical or edible composition, which comprises a therapeutically effective amount of the Cordylutene, or a pharmaceutically, cosmetically or edibly acceptable salt thereof, or a mixture thereof, together with a pharmaceutically, cosmetically or edibly acceptable carrier.

In some specific examples of the invention, the Cordylutene is a compound selected from the group consisting of the compounds having the structures below:

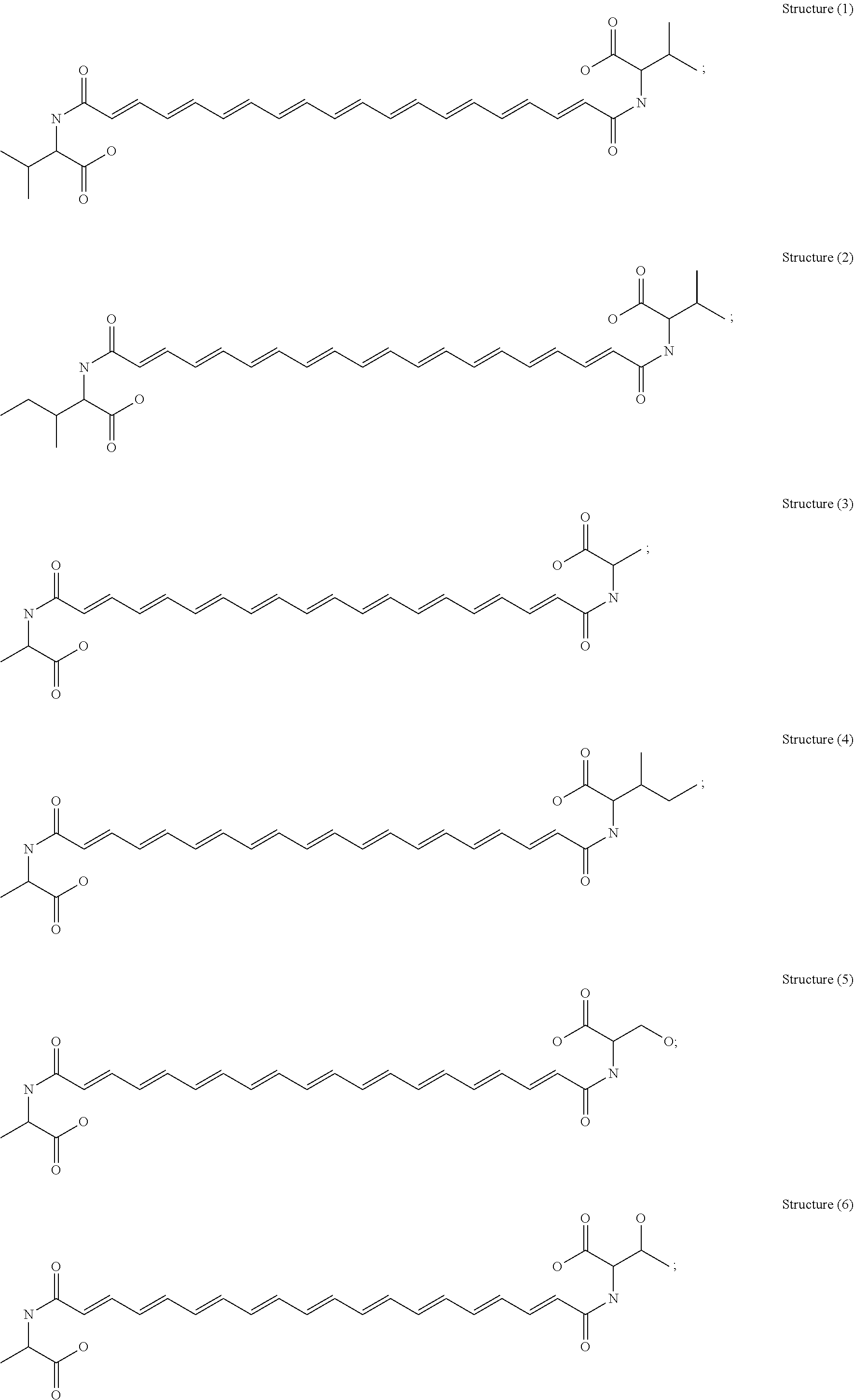

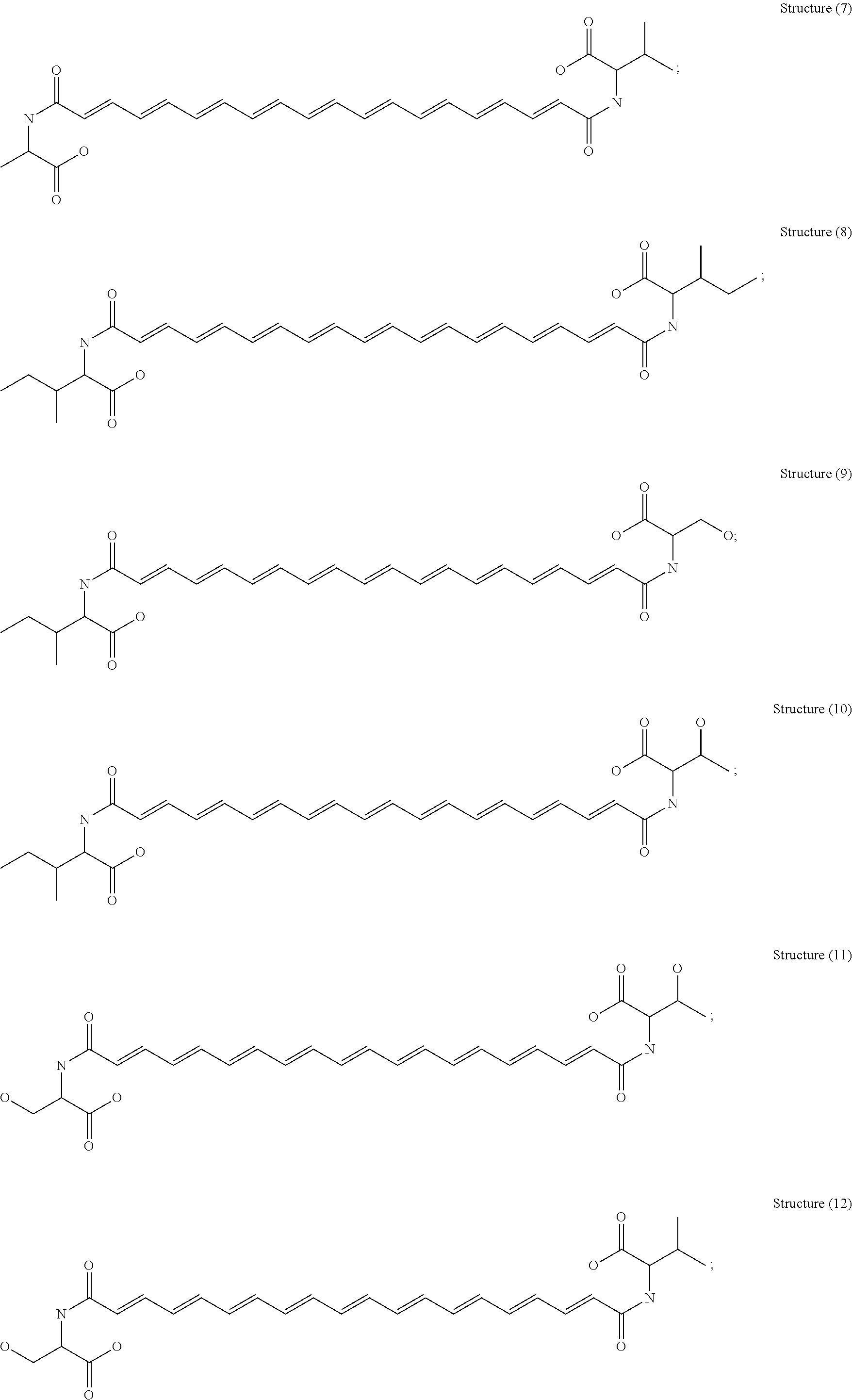

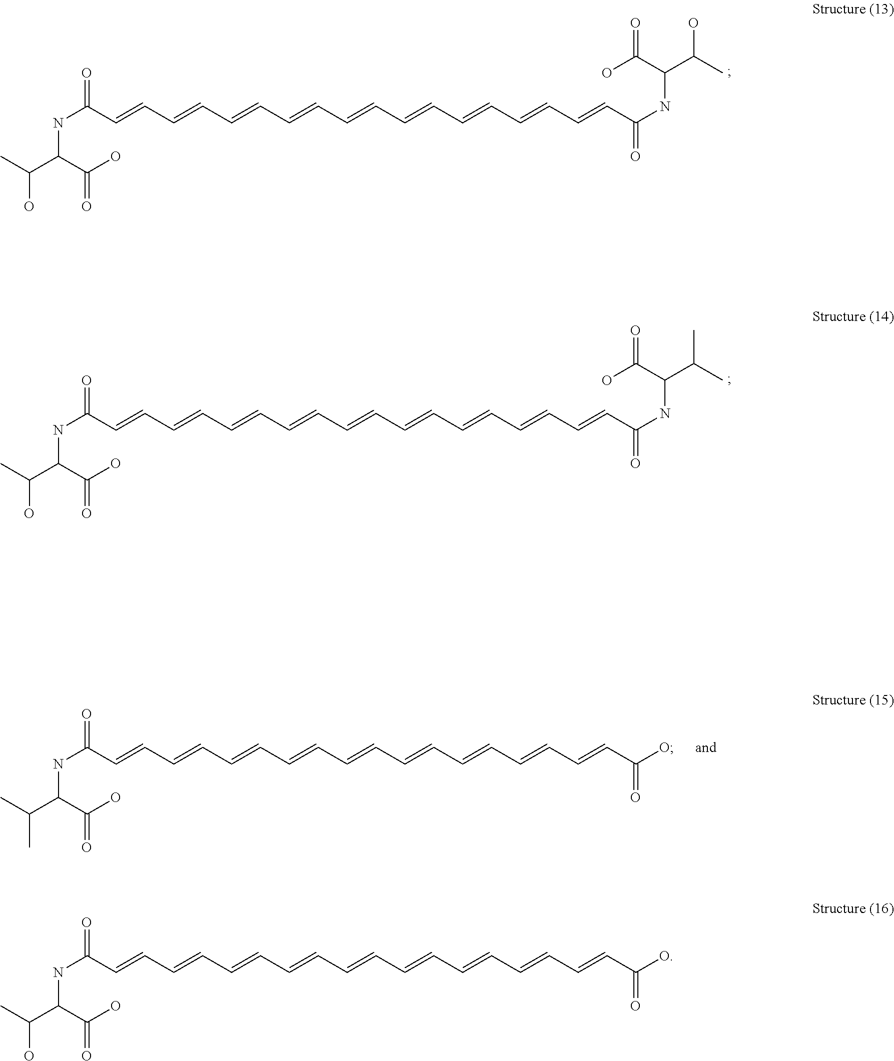

In one embodiment of the invention, the ocular disease is caused by prolonged exposure to a light source containing blue wavelengths, which is selected from the group consisting of dry eye disease, cataracts, glaucoma, and macular degeneration.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1A shows the effects of Cordylutene A on ARPE-19 cell viability under oxidative stress. ***p<0.001 vs. H₂O₂ group (Tukey's post hoc test after one-way ANOVA), data are expressed as mean±SD (n=5).

FIG. 1B shows the effects of Cordylutene A on intracellular ROS generation. ***p<0.001 vs. H₂O₂ group (Tukey's post hoc test after one-way ANOVA), data are expressed as mean±SD (n=5).

FIG. 1C shows the effects of Cordylutene A on malondialdehyde (MDA) levels in ARPE-19. ***p<0.001 vs. H₂O₂ group (Tukey's post hoc test after one-way ANOVA), data are expressed as mean±SD (n=5).

FIG. 1D shows the effects of Cordylutene A on SOD activity in ARPE-19. ***p<0.001 vs. H₂O₂ group (Tukey's post hoc test after one-way ANOVA), data are expressed as mean SD (n=5).

FIG. 2A shows the effects of Cordylutene A on the viability of HaCaT cells after UVB exposure. ***p<0.001 vs. UVB group; **p<0.01 vs. UVB group (Tukey's post hoc test after one-way ANOVA), data are shown as mean±SD (n=5).

FIG. 2B shows the effects of Cordylutene A on MDA level in UVB-exposed HaCaT cells. ***p<0.001 vs. UVB group; **p<0.01 vs. UVB group (Tukey's post hoc test after one-way ANOVA), data are shown as mean±SD (n=5). ***p<0.001 vs. H₂O₂ group (Tukey's post hoc test after one-way ANOVA), data are shown as mean±SD (n=5).

FIG. 2C shows the effects of Cordylutene A on SOD enzyme activity in HaCaT cells exposed to UVB. ***p<0.001 vs. UVB group; **p<0.01 vs. UVB group (Tukey's post hoc test after one-way ANOVA), data are shown as mean±SD (n=5). ***p<0.001 vs. H₂O₂ group (Tukey's post hoc test after one-way ANOVA), data are shown as mean±SD (n=5).

FIG. 3A shows the effects of Cordylutene A on HLE-B3 cell viability under oxidative stress. ***p<0.001 vs. H₂O₂ group (Tukey's post hoc test after one-way ANOVA), data are shown as mean SD (n=5).

FIG. 3B shows the effects of Cordylutene A on intracellular ROS levels in HLE-B3 cells. ***p<0.001 vs. H₂O₂ group (Tukey's post hoc test after one-way ANOVA), data are shown as mean SD (n=5).

FIG. 4A shows the effects of Cordylutene A on IL-1β mRNA expression in HCECs. ***p<0.001, **p<0.01 vs. hyperosmotic model group (Tukey's post hoc test after one-way ANOVA), data are expressed as mean±SD (n=5).

FIG. 4B shows the effects of Cordylutene A on IL-6 mRNA expression in HCECs. ***p<0.001, **p<0.01 vs. hyperosmotic model group (Tukey's post hoc test after one-way ANOVA), data are expressed as mean±SD (n=5).

FIG. 4C shows the effects of Cordylutene A on TNF-α mRNA expression in HCECs. ***p<0.001, **p<0.01 vs. hyperosmotic model group (Tukey's post hoc test after one-way ANOVA), data are expressed as mean±SD (n=5).

FIG. 4D shows the effects of Cordylutene A on IL-1β protein levels under hyperosmotic stress. ***p<0.001, **p<0.01 vs. hyperosmotic model group (Tukey's post hoc test after one-way ANOVA), data are expressed as mean±SD (n=5).

FIG. 4E shows the effects of Cordylutene A on IL-6 protein levels under hyperosmotic stress. ***p<0.001, **p<0.01 vs. hyperosmotic model group (Tukey's post hoc test after one-way ANOVA), data are expressed as mean±SD (n=5).

FIG. 4F shows the effects of Cordylutene A on TNF-α protein levels under hyperosmotic stress. ***p<0.001, **p<0.01 vs. hyperosmotic model group (Tukey's post hoc test after one-way ANOVA), data are expressed as mean±SD (n=5).

FIG. 5A shows the effects of Cordylutene A against A2E-induced phototoxicity. ***p<0.001; **p<0.01 vs. A2E group (one-way ANOVA followed by Tukey's post hoc test), data are presented as mean±SD (n=3).

FIG. 5B shows the effects of Cordylutene A and lutein on intracellular ROS levels in ARPE-19 cells under A2E-induced blue light stress. ***p<0.001; **p<0.01 vs. A2E group (one-way ANOVA followed by Tukey's post hoc test), data are presented as mean±SD (n=3).

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an” and “the” include plural references unless explicitly indicated otherwise. Thus, for example, reference to “a sample” includes a plurality of such samples and their equivalents known to those skilled in the art.

The present invention provides a new compound, named as Cordylutene, which has the structure of general Formula (I) below, or a pharmaceutically, cosmetically or edibly acceptable salt thereof:

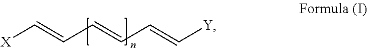

wherein:
X and Y are the same or different, independently selected from the group consisting of aldehyde, ketone, carboxylic acid, ester, amide, acyl halide, acid anhydride, imide, and thioester; and
n is an integer from 5 to 9.

In one embodiment of the present invention, X is a functional group represented by Formula A:

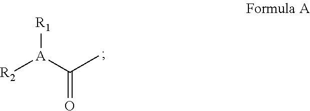

and Y is a functional group represented by Formula B:

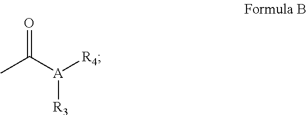

wherein:
A is a carbon, sulfur, oxygen, nitrogen, or hydrogen; and

R₁, R₂, R₃ and R₄ are the same, different or absent, independently selected from the group consisting of hydrogen, halogen, hydroxyl, cyano, nitro, thiol, alkyl, aryl, alkenyl, alkynyl, alkoxy, aryloxy, acyl, sulfonyl, amino, and heterocyclic groups. In another embodiment of the present invention, X is a functional group represented by Formula C:

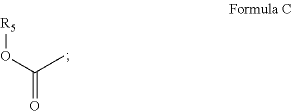

and Y is a functional group represented by Formula D:

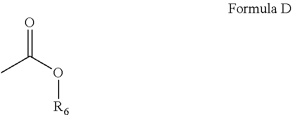

wherein:
R₅ and R₆ are the same or different, independently selected from the group consisting of hydrogen, and a substituted or unsubstituted organic or inorganic radical, wherein the radical is selected from alkyl, aryl, alkenyl, alkynyl, alkoxy, aryloxy, acyl, sulfonyl, amino, heterocyclic groups, halogen, hydroxyl, cyano, or nitro.

In a further embodiment of the present invention, X is a functional group represented by Formula E:

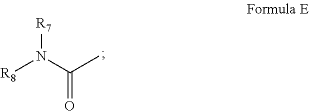

and Y is a functional group represented by Formula F:

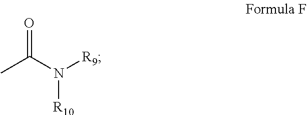

wherein:
R₇ to R₁₀ are the same or different, independently selected from the group consisting of hydrogen, and a substituted or unsubstituted organic or inorganic radical, wherein the radical is selected from alkyl, aryl, alkenyl, alkynyl, alkoxy, aryloxy, acyl, sulfonyl, amino, heterocyclic groups, halogen, hydroxyl, cyano and nitro.

In the present invention, each of R₁-R₁₀ may be:
(i) an alkyl group, selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, and tetracosyl group, which may be optionally further substituted with hydroxyl, thiol, amino, nitro, cyano group, or halogen;
(ii) an aryl group, selected from the group consisting of a phenyl and a substituted phenyl group, which may be optionally further substituted with alkyl, hydroxyl, alkoxy, aryloxy, amino, nitro, cyano group, or other heteroatom substituent;
(iii) an alkenyl group, which is vinyl or allyl group, which may be optionally further substituted with a hydroxyl, phenyl, halogen, nitro, amino, carboxyl, ester, ether, thioether, or sulfonyl group;
(iv) an alkynyl group, which is ethynyl or propynyl group, which may be optionally further substituted with a hydroxyl, phenyl, halogen, nitro, amino, carboxyl, ester, ether, thioether, or sulfonyl group;
(v) an alkoxy or aryloxy group, which has an oxygen atom bonded to an alkyl or aryl group, wherein the alkyl group may be optionally further substituted with a hydroxyl, thiol, amino, nitro, cyano group, or other heteroatom substituents; and wherein the aryl group may be optionally further substituted with alkyl, hydroxyl, alkoxy, aryloxy, amino, nitro, cyano group, or halogen;
(vi) an acyl group, which has a carbonyl group bonded to an alkyl or aryl group, wherein the alkyl group may be optionally further substituted with hydroxyl, thiol, amino, nitro, cyano group, or other heteroatom substituent; and wherein the aryl group may be optionally further substituted with alkyl, hydroxyl, alkoxy, aryloxy, amino, nitro, cyano group, or halogen;
(vii) a sulfonyl group, which is bonded to an alkyl or aryl group, wherein the alkyl group may be optionally further substituted with hydroxyl, thiol, amino, nitro, cyano groups, or halogens; and wherein the aryl group may be optionally further substituted with alkyl, hydroxyl, alkoxy, aryloxy, amino, nitro, cyano group, or halogen;
(viii) an amino group, selected from the group consisting of primary amino group, secondary amino group, and tertiary amino group, which comprises a nitrogen atom bonded to one or more alkyl or aryl groups, wherein the alkyl group may be optionally further substituted with hydroxyl, thiol, amino, nitro, a cyano group, a halogen, or other heteroatom substituent; and wherein the aryl group may be optionally further substituted with alkyl, hydroxyl, alkoxy, aryloxy, amino, nitro, cyano group, or halogen; and
(ix) a heterocyclic group, which is pyridyl, furyl, thienyl, imidazolyl, thiazolyl, oxazolyl, pyrrolyl, piperidyl, or morpholinyl group, wherein the heterocyclic group may be optionally further substituted with alkyl, hydroxyl, alkoxy, aryloxy, amino, nitro, cyano group, halogen, or other heteroatom substituent.

In a yet embodiment of the present invention, X is a functional group represented by Formula G:

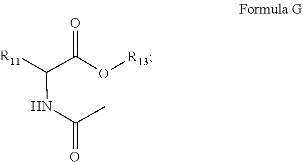

and Y is a functional group represented by Formula H:

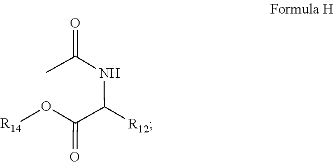

wherein:
R₁₁ to R₁₄ are the same or different, independently selected from the group consisting of hydrogen, and a substituted or unsubstituted organic or inorganic radical, wherein the radical is selected from alkyl, aryl, alkenyl, alkynyl, alkoxy, aryloxy, acyl, sulfonyl, amino, heterocyclic groups, halogen, hydroxyl, cyano and nitro.

In some examples of the present invention, R₁₁ and R₁₂ are the same or different, each independently is an unsubstituted or substituted side chain derived from amino acid, which is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, wherein the side chain is a substituent selected from the group consisting of methyl, isopropyl, sec-butyl, 2-butyl, thioether, benzyl, indole, hydroxymethyl, 1-hydroxyethyl, thiol, part of the cyclic structure, amide, carboxyl, long aliphatic chains, guanidinium, and imidazole.

As used herein, the term “aldehyde” refers to an organic compound characterized by the presence of a formyl group, which has a carbonyl group (i.e., a carbon atom double-bonded to an oxygen atom) bonded to a hydrogen atom.

As used herein, the term “ketone” refers to a carbonyl group bonded to a diverse group, such as alkyl, aryl, alkenyl, alkynyl, or heterocyclic groups, which may be optionally further substituted with one or more functional groups, such as hydroxyl, halogen, nitro, amino, carboxyl, ester, ether, thioether, and sulfonyl groups.

As used herein, the term “carboxylic acid” refers to a carbonyl group bonded to a hydroxyl group.

As used herein, the term “ester” refers to a carbonyl group bonded to an oxygen atom, wherein the oxygen atom may be optionally further bonded to one or more functional groups, such as alkyl, aryl, alkenyl, alkynyl, and heterocyclic groups, which may be optionally further substituted with one or more functional groups, such as hydroxyl, halogen, nitro, amino, carboxyl, ester, ether, thioether, or sulfonyl groups.

As used herein, the term “amide” refers to a carbonyl group bonded to a nitrogen atom, wherein the nitrogen atom may be optionally further bonded to a hydrogen atom or one or more functional group, such as alkyl, aryl, alkenyl, alkynyl, or heterocyclic groups, which may be optionally further substituted with one or more functional groups, such as hydroxyl, phenyl, halogen, nitro, amino, carboxyl, ester, ether, thioether, or sulfonyl groups.

As used herein, the term “acyl halide” refers to a carbonyl group bonded to a halogen atom, such as fluorine, chlorine, bromine, or iodine.

As used herein, the term “acid anhydride” refers to a carbonyl group bonded to another carbonyl via an oxygen atom, wherein the carbonyl may be substituted with one or more functional groups, such as alkyl, aryl, alkenyl, alkynyl, or heterocyclic groups. These groups may be optionally further substituted with one or more functional groups, such as hydroxyl, phenyl, halogen, etc.

As used herein, the term “imide” refers to a carbonyl group bonded to another carbonyl group via a nitrogen atom, wherein the nitrogen atom may be optionally further bonded to one or more functional group, such as alkyl, aryl, alkenyl, alkynyl, or heterocyclic groups.

As used herein, the term “thioester” refers to a carbonyl group bonded to a sulfur atom, which may be optionally further bonded to one or more functional groups, such as alkyl, aryl, alkenyl, alkynyl, or heterocyclic groups, which may be optionally further substituted with one or more functional groups, such as hydroxyl, phenyl, halogen, nitro, amino, carboxyl, ester, ether, thioether, or sulfonyl groups.

As used herein, the term “halogen” includes fluorine, chlorine, bromine, or iodine.

As used herein, the term “alkyl” contemplates both straight and branched chain alkyl radicals. Preferred alkyl groups include those containing from 1-24 carbon atoms and includes methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, and tetracosyl groups; branched alkyls, such as iso-propyl, iso-butyl, tert-butyl and the like; or cyclic alkyl radicals, such as cyclopropyl, cyclobutyl, cyclohexyl and the like.

As used herein, the term “aryl” refers to a phenyl or substituted phenyl group with one or more substituents, such as alkyl, hydroxyl, alkoxy, aryloxy, amino, nitro, cyano, or halogen groups.

As used herein, the term “alkenyl” contemplates both straight and branched chain alkene radicals, which refers to a carbon group containing a double bond, such as vinyl or allyl.

As used herein, the term “alkynyl” contemplates both straight and branched chain alkyne radicals, which refers to an alkynyl group containing a triple bond, such as ethynyl or propynyl.

As used herein, the term “heterocyclic group” refers to a stable 3- to 10-membered, aromatic or non-aromatic cyclic radical, which consists of carbon atoms and at least one heteroatom selected from the group consisting of nitrogen, oxygen, and sulfur.

In one particular embodiment of the present invention, the Cordylutene has the structure of Formula (II) below:

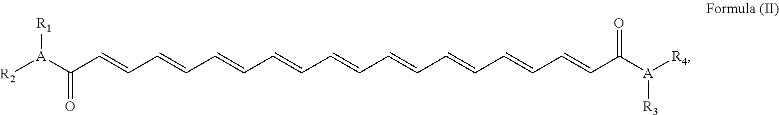

wherein
A is selected from the group consisting of carbon, sulfur, oxygen, nitrogen, and hydrogen; and
R₁ to R₄ are the same, different or absent, independently selected from the group consisting of hydrogen, halogen, hydroxyl, cyano, nitro, thiol, alkyl, aryl, alkenyl, alkynyl, alkoxy, aryloxy, acyl, sulfonyl, amino, and heterocyclic groups.

In some examples of the compound having the structure of Formula (II), R₁, R₂, R₃, and R₄ are the same, different or absent, each is independently an alkyl group, such as methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, or tetracosyl group, which may be optionally further substituted with hydroxyl, phenyl, thiol, amino, nitro, cyano group, or halogen.

In some examples of the compound having the structure of Formula (II), R₁, R₂, R₃, and R₄ are the same different or absent, each is independently an aryl group, such as a phenyl or substituted phenyl group, which may be optionally further substituted with hydroxyl, halogen, nitro, amino, carboxyl, ester, ether, thioether, or sulfonyl group.

In some examples of the compound having the structure of Formula (II), R₁, R₂, R₃, and R₄ are the same, different or absent, each is independently an alkenyl group, such as vinyl, allyl, ethynyl, or propynyl group, which may be optionally further substituted with hydroxyl, phenyl, halogen, nitro, amino, carboxyl, ester, ether, thioether, or sulfonyl group.

In some examples of the compound having the structure of Formula (II), R₁, R₂, R₃, and R₄ are the same, different or absent, each independently is an alkoxy or aryloxy groups, which is an oxygen bonded to an alkyl group or an aryl group, wherein the alkyl group or aryl group may be optionally further substituted with hydroxyl, halogen, nitro, amino, carboxyl, ester, ether, thioether, or sulfonyl group.

In some examples of the compound having the structure of Formula (II), R₁, R₂, R₃, and R₄ are the same, different or absent, each independently is a group containing a heteroatom, such as methoxy, ethoxy, propoxy, methylthio, ethylthio, amino group, fluoro, chloro, bromo, or iodo; or a heterocyclic group containing nitrogen, oxygen, or sulfur atom, which may be optionally further substituted with hydroxyl, alkoxy, aryloxy, amino, nitro, cyano, or other heteroatom substituent.

In another embodiment of the present invention, the Cordylutene has the structure of Formula (III) below:

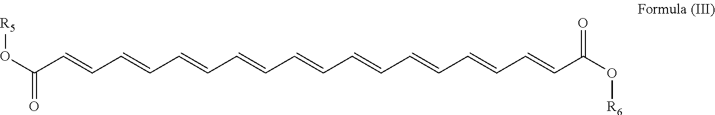

wherein
R₅ and R₆ are the same or different, independently selected from the group consisting of hydrogen, and a substituted or unsubstituted organic or inorganic radical, wherein the radical is selected from the group consisting of alkyl, aryl, alkenyl, alkynyl, alkoxy, aryloxy, acyl, sulfonyl, amino, heterocyclic groups, halogen, hydroxyl, cyano, and nitro.

In some examples of the compound having the structure of Formula (III), each of R₅ and R₆ is an alkyl group, such as methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, or tetracosyl group, which may be optionally substituted with hydroxyl, thiol, amino, nitro, cyano, or halogen.

In some examples of the compound having the structure of Formula (III), each of R₅ and R₆ is an aryl group, such as phenyl or substituted phenyl group, which may be optionally further substituted with alkyl, hydroxyl, alkoxy, aryloxy, amino, nitro, cyano, or other heteroatom substituent.

In some examples of the compound having the structure of Formula (III), each of R₅ and R₆ is an alkenyl group, such as vinyl, allyl, ethynyl, or propynyl groups, which may be optionally further substituted with hydroxyl, phenyl, halogen, nitro, amino, carboxyl, ester, ether, thioether, or sulfonyl group.

In some examples of the compound having the structure of Formula (III), each of R₅ and R₆ is an alkoxy or aryloxy group, which is an oxygen atom bonded to an alkyl or aryl group, wherein the alkyl group may be optionally further substituted with hydroxyl, thiol, amino, nitro, cyano, or other heteroatom substituent; and wherein the aryl group may be optionally further substituted with alkyl, hydroxyl, alkoxy, aryloxy, amino, nitro, cyano groups, or halogen.

In some examples of the compound having the structure of Formula (III), each of R₅ and R₆ is a carbonyl group bonded to an alkyl or aryl group, wherein the alkyl group may be optionally further substituted with hydroxyl, thiol, amino, nitro, cyano group, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (III), each of R₅ and R₆ is a sulfonyl group bonded to an alkyl or aryl group, wherein the alkyl group may be optionally further substituted with hydroxyl, thiol, amino, nitro, cyano groups, or halogens; and wherein the aryl group may be optionally further substituted with alkyl, hydroxyl, alkoxy, aryloxy, amino, nitro, cyano group, or halogen.

In some examples of the compound having the structure of Formula (III), each of R₅ and R₆ is an amino group, such as a primary amino group, secondary amino group, or tertiary amino group; which comprises a nitrogen atom bonded to one or more alkyl or aryl groups, wherein the alkyl group may be optionally further substituted with hydroxyl, thiol, amino, nitro, cyano group, halogen, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (III), each of R₅ and R₆ is a heterocyclic group, such as pyridyl, furyl, thienyl, imidazolyl, thiazolyl, oxazolyl, pyrrolyl, piperidyl, or morpholinyl, wherein the heterocyclic group may be optionally further substituted with alkyl, hydroxyl, alkoxy, aryloxy, amino, nitro, cyano group, halogen, or other heteroatom substituent.

In one more embodiment of the present invention, the Cordylutene is represented by Formula (IV) below:

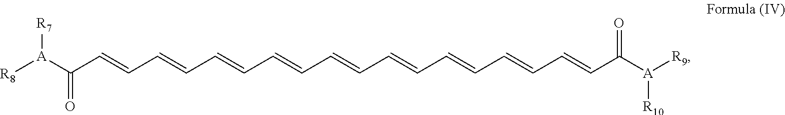

wherein:
R₇ to R₁₀ are the same or different, independently selected from the group consisting of hydrogen, and a substituted or unsubstituted organic or inorganic radical, wherein the radical is alkyl, aryl, alkenyl, alkynyl, alkoxy, aryloxy, acyl, sulfonyl, amino, heterocyclic groups, halogen, hydroxyl, cyano, or nitro.

In some examples of the compound having the structure of Formula (IV), each of R₇, R₈, R₉ and R₁₀ is an alkyl group, such as methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, or tetracosyl group, which may be optionally further substituted with hydroxyl, thiol, amino, nitro, cyano group, or halogen.

In some examples of the compound having the structure of Formula (IV), each of R₇, R₈, R₉ and R₁₀ is an aryl group, such as phenyl or substituted phenyl group, which may be optionally further substituted with alkyl, hydroxyl, alkoxy, aryloxy, amino, nitro, cyano group, or other heteroatom substituent.

In some examples of the compound having the structure of Formula (IV), each of R₇, R₈, R₉ and R₁₀ is an alkenyl group, such as vinyl, allyl, ethynyl, or propynyl group, which may be optionally further substituted with hydroxyl, phenyl, halogen, nitro, amino, carboxyl, ester, ether, thioether, or sulfonyl group.

In some examples of the compound having the structure of Formula (IV), each of R₇, R₈, R₉ and R₁₀ is an alkoxy or aryloxy group, which is an oxygen atom bonded to an alkyl or aryl group, wherein the alkyl group may be optionally further substituted with hydroxyl, thiol, amino, nitro, cyano, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (IV), each of R₇, R₈, R₉ and R₁₀ is an acyl group, which is a carbonyl group bonded to an alkyl or aryl group, wherein the alkyl group may be optionally further substituted with hydroxyl, thiol, amino, nitro, cyano group, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (IV), each of R₇, R₈, R₉ and R₁₀ is a sulfonyl group bonded to an alkyl or aryl group, wherein the alkyl group may be optionally further substituted with hydroxyl, thiol, amino, nitro, cyano group, or halogen; and wherein the aryl group may be optionally further substituted with alkyl, hydroxyl, alkoxy, aryloxy, amino, nitro, cyano group, or halogen.

In some examples of the compound having the structure of Formula (IV), each of R₇, R₈, R₉ and R₁₀ is an amino group, such as primary amino group, secondary amino group, or tertiary amino group; which comprises a nitrogen atom bonded to an alkyl or aryl group, wherein the alkyl group may be optionally further substituted with hydroxyl, thiol, amino, nitro, cyano group, halogen, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (IV), each of R₇, R₈, R₉ and R₁₀ is a heterocyclic group, comprising pyridyl, furyl, thienyl, imidazolyl, thiazolyl, oxazolyl, pyrrolyl, piperidyl, or morpholinyl group, wherein the heterocyclic group may be optionally further substituted with alkyl, hydroxyl, alkoxy, aryloxy, amino, nitro, cyano group, halogen, or other heteroatom substituent.

In the other embodiment of the present invention, the Cordylutene has the structure of Formula (V) below:

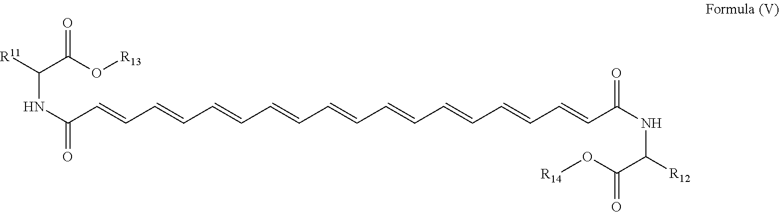

wherein:
R₁₁ to R₁₄ are the same or different, independently selected from the group consisting of hydrogen, and a substituted or unsubstituted organic or inorganic radical, wherein the radical is alkyl, aryl, alkenyl, alkynyl, alkoxy, aryloxy, acyl, sulfonyl, amino, heterocyclic groups, halogen, hydroxyl, cyano, or nitro.

In some examples of the compound having the structure of Formula (V), each of R₁₁, R₁₂, R₁₃ and R₁₄ is an alkyl group, such as methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosyl, docosyl, tricosyl, and tetracosyl groups, which may be optionally further be substituted with hydroxyl, thiol, amino, nitro, cyano group, or halogen.

In some examples of the compound having the structure of Formula (V), each of R₁₁, R₁₂, R₁₃ and R₁₄ is an aryl group, comprising phenyl or substituted phenyl group, which may be optionally further substituted with alkyl, hydroxyl, alkoxy, aryloxy, amino, nitro, cyano group, or other heteroatom substituent.

In some examples of the compound having the structure of Formula (V), each of R₁₁, R₁₂, R₁₃ and R₁₄ is an alkenyl group, such as vinyl, allyl, ethynyl, or propynyl group, which may be optionally further substituted with hydroxyl, phenyl, halogen, nitro, amino, carboxyl, ester, ether, thioether, or sulfonyl group.

In some examples of the compound having the structure of Formula (V), each of R₁₁, R₁₂, R₁₃ and R₁₄ is an alkoxy or aryloxy group, which is an oxygen atom bonded to an alkyl or aryl group, wherein the alkyl group may be optionally further substituted with hydroxyl, thiol, amino, nitro, cyano, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (V), each of R₁₁, R₁₂, R₁₃ and R₁₄ is an acyl group, which is a carbonyl group bonded to an alkyl or aryl group, wherein the alkyl group may be optionally further with hydroxyl, thiol, amino, nitro, cyano, or other heteroatom substituent; and wherein the aryl group may be further substituted with alkyl, hydroxyl, alkoxy, aryloxy, amino, nitro, cyano group, or halogen.

In some examples of the compound having the structure of Formula (V), each of R₁₁, R₁₂, R₁₃ and R₁₄ is a sulfonyl group bonded to an alkyl or aryl group, wherein the alkyl group may be optionally further substituted with hydroxyl, thiol, amino, nitro, cyano group, or halogen; and wherein the aryl group may be optionally further substituted with alkyl, hydroxyl, alkoxy, aryloxy, amino, nitro, cyano group, or halogen.

In some examples of the compound having the structure of Formula (V), each of R₁₁, R₁₂, R₁₃ and R₁₄ is an amino group, such as primary amino group, secondary amino group, or tertiary amino group, which comprises a nitrogen atom bonded to one or more alkyl or aryl group, wherein the alkyl group may be optionally further substituted with hydroxyl, thiol, amino, nitro, cyano group, halogen, or other heteroatom substituent; and wherein the aryl group may be optionally further substituted with alkyl, hydroxyl, alkoxy, aryloxy, amino, nitro, cyano group, or halogen.

In some examples of the compound having the structure of Formula (V), each of R₁₁, R₁₂, R₁₃ and R₁₄ is a heterocyclic group, such as pyridyl, furyl, thienyl, imidazolyl, thiazolyl, oxazolyl, pyrrolyl, piperidyl, or morpholinyl, wherein the heterocyclic group may be optionally further substituted with alkyl, hydroxyl, alkoxy, aryloxy, amino, nitro, cyano groups, halogen, or other heteroatom substituent.

In some examples of the compound having the structure of Formula (V), R₁₁ and R₁₂ are the same or different, each of R₁₁ and R₁₂ is a side chain derived from common amino acids when R₁₃ and R₁₄ are unsubstituted, wherein the common amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine; wherein the side chains include methyl, isopropyl, sec-butyl, 2-butyl, thioether, benzyl, indole, hydroxymethyl, 1-hydroxyethyl, thiol, part of the cyclic structure, amide, carboxyl, long aliphatic chains, guanidinium, and imidazole.

In particular examples of the present invention, the Cordylutene has the structure of Formula (I), or a pharmaceutically, cosmetically or edibly acceptable salt thereof,
wherein
X is a functional group represented by Formula A:

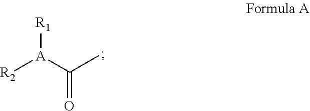

Y is a functional group represented by Formula B:

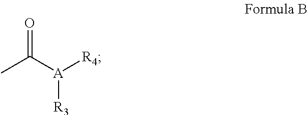

in which:
A is a sulfur, oxygen or nitrogen; and
R₁, R₂, R₃ and R₄ are the same, different or absent, independently selected from the group consisting of hydrogen, ethyl and amino acid selected from the group consisting of alanine (Ala), isoleucine (Ile), serine (Ser), threonine (Thr), and valine (Val).

In particular, the Cordylutene is selected from the group consisting of the compounds shown in Table 1 below, the preparation for which is illustrated in the examples.

TABLE 1
Cordylutenes as prepared in the present invention.

| Structure | Formula (I) | | | Formula A | | | Formula B | | |
|---|---|---|---|---|---|---|---|---|---|
| No. | n | X | Y | A | R₁ | R₂ | A | R₃ | R₄ |
| 1 | 7 | amide | amide | N | -Val | H | N | -Val | H |
| 2 | 7 | amide | amide | N | -Val | H | N | -Ile | H |
| 3 | 7 | amide | amide | N | -Ala | H | N | -Ala | H |
| 4 | 7 | amide | amide | N | -Ala | H | N | -Ile | H |
| 5 | 7 | amide | amide | N | -Ala | H | N | -Ser | H |
| 6 | 7 | amide | amide | N | -Ala | H | N | -Thr | H |
| 7 | 7 | amide | amide | N | -Ala | H | N | -Val | H |
| 8 | 7 | amide | amide | N | -Ile | H | N | -Ile | H |
| 9 | 7 | amide | amide | N | -Ile | H | N | -Ser | H |
| 10 | 7 | amide | amide | N | -Ile | H | N | -Thr | H |
| 11 | 7 | amide | amide | N | -Ser | H | N | -Thr | H |
| 12 | 7 | amide | amide | N | -Ser | H | N | -Val | H |
| 13 | 7 | amide | amide | N | -Thr | H | N | -Thr | H |
| 14 | 7 | amide | amide | N | -Thr | H | N | -Val | H |
| 15 | 7 | amide | carboxylic | N | -Val | H | O | H | — |
| 16 | 7 | amide | carboxylic | N | -Thr | H | O | H | — |
| 24 | 7 | carboxylic | carboxylic | O | H | — | O | H | — |
| 27 | 6 | carboxylic | carboxylic | O | H | — | O | H | — |
| 28 | 7 | ester | ester | O | CH₂CH₃ | — | O | CH₂CH₃ | — |
| 29 | 7 | thioester | thioester | S | CH₂CH₃ | — | S | CH₂CH₃ | — |
| 30 | 7 | amide | amide | N | -Ser | H | N | -Ser | H |

In particular examples of the present invention, the Cordylutene is one selected from the group consisting of the compounds of the following structures:

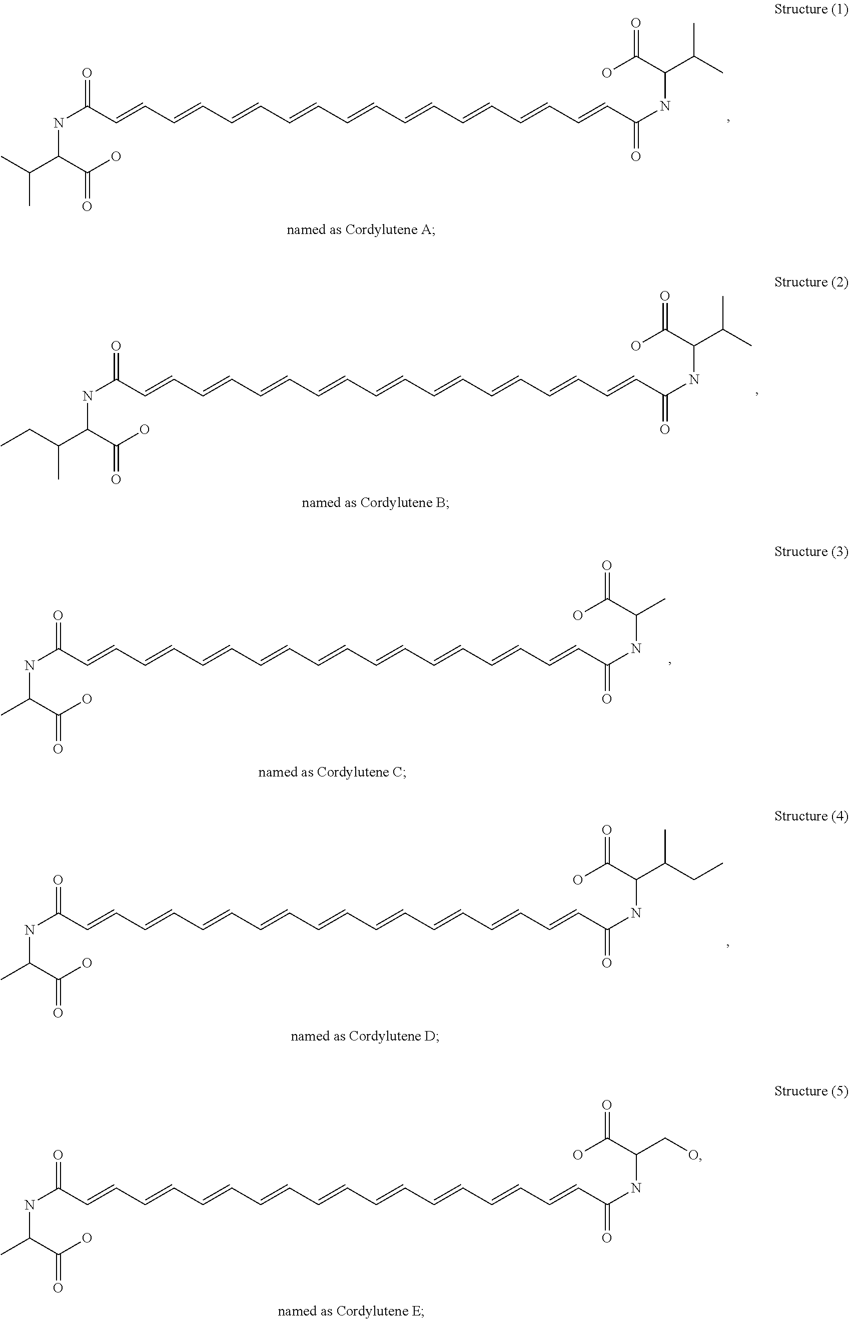

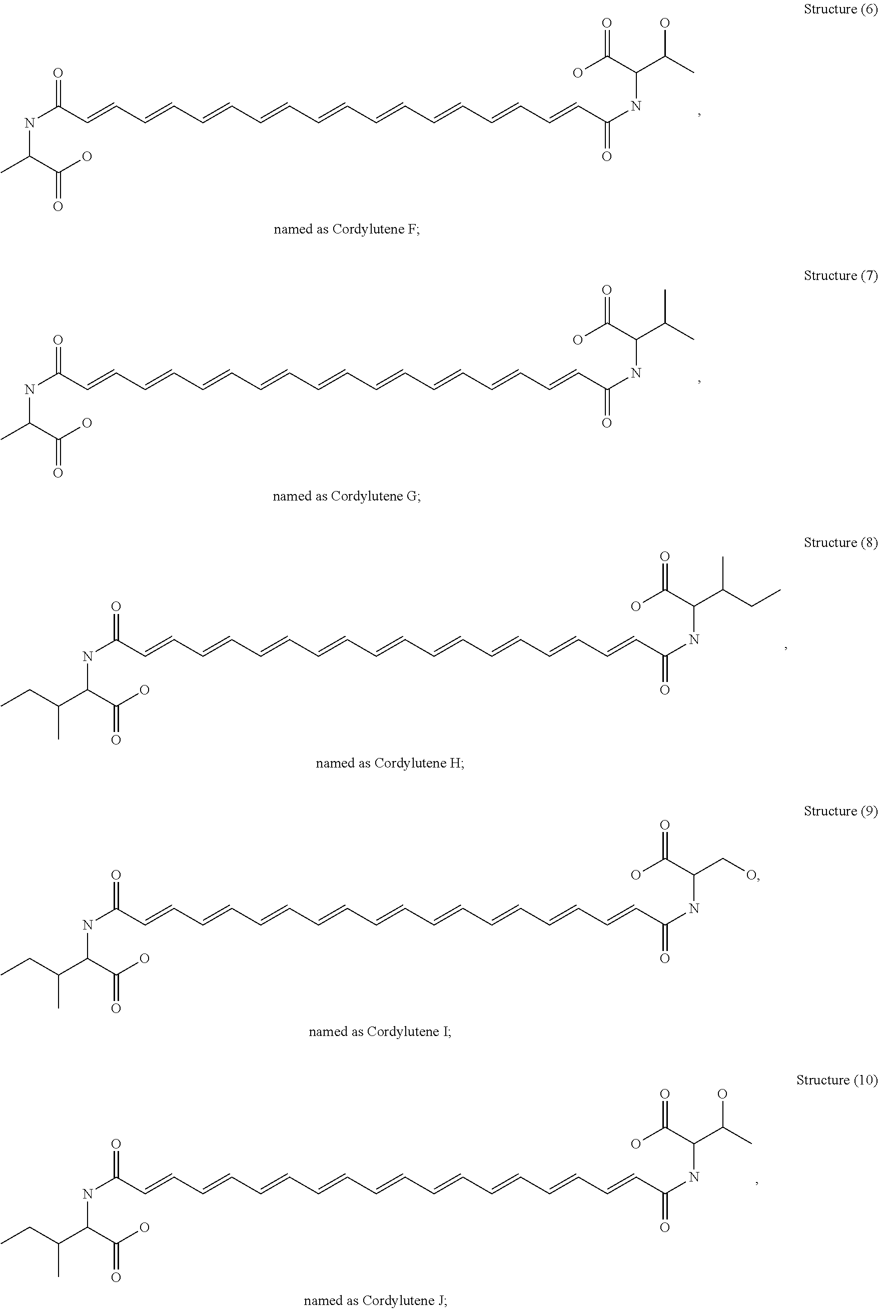

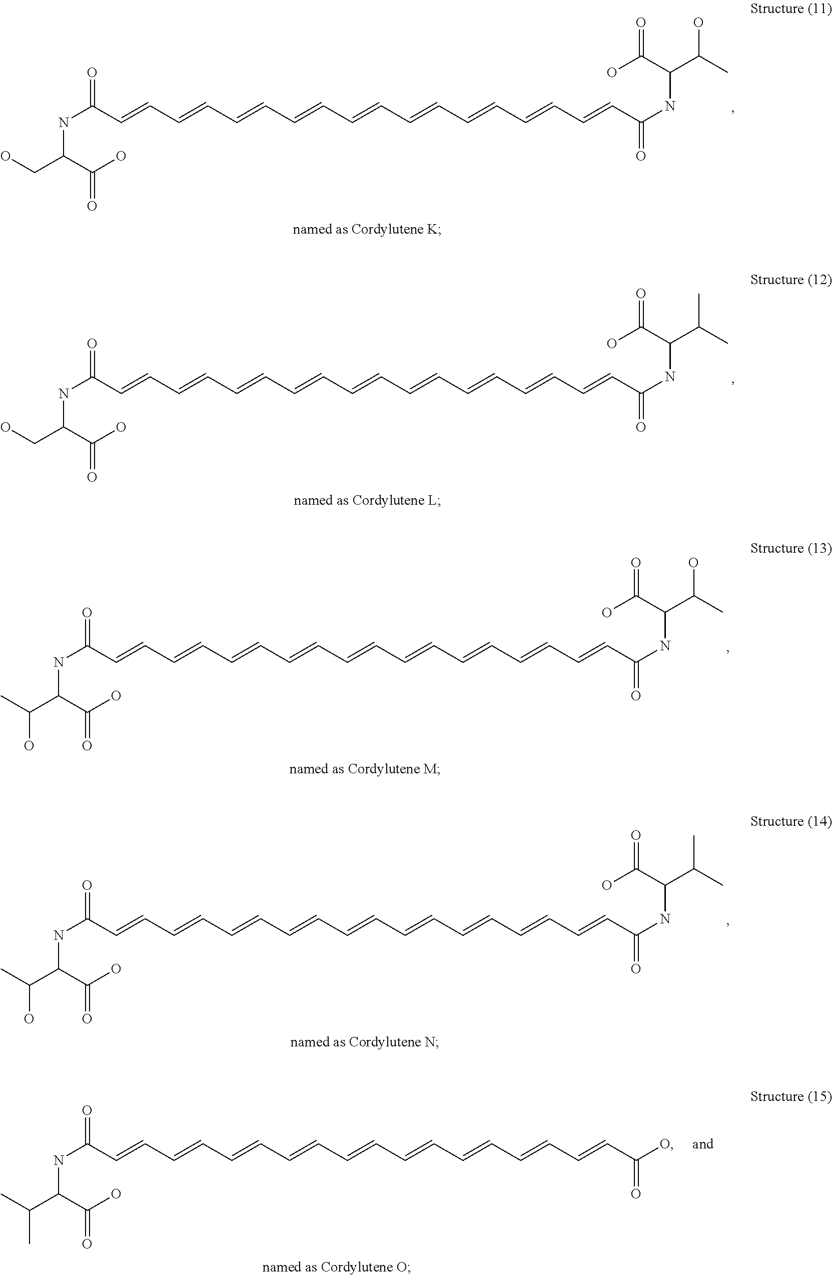

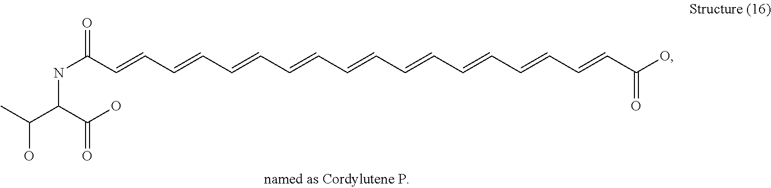

In one particular example of the present invention, the Cordylutene derivative is the compound of Structure (30):

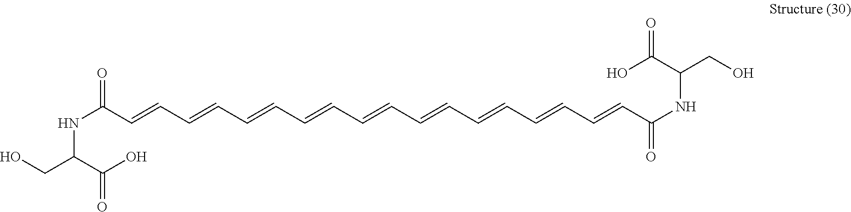 [SMILES: O=C(/C=C/C=C/C=C/C=C/C=C/C=C/C=C/C=C/C=C/C(=O)NC(CO)C(=O)O)NC(CO)C(=O)O]

It is ascertained in the examples that the Cordylutenes exhibit the effects in preventing or treating an ocular disease caused by prolonged exposure to a light source containing blue wavelengths, which is selected from the group consisting of dry eye disease, cataracts, glaucoma, and macular degeneration.

Accordingly, the present invention provides a method for preventing or treating an ocular disease in a subject comprising administrating the subject a pharmaceutical, cosmetical or edible composition comprising a therapeutically effective amount of the Cordylutene, or a pharmaceutically, cosmetically or edibly acceptable salt thereof, or a mixture thereof. The present invention also provides a use of Cordylutene for manufacturing a medicament for preventing or treating an ocular disease caused by prolonged exposure to a light source containing blue wavelengths, which is selected from the group consisting of dry eye disease, cataracts, glaucoma, and macular degeneration.

In addition, the present invention provides a pharmaceutically, cosmetically or edibly acceptable composition comprising a therapeutically effective amount of Cordylutene as defined above or a pharmaceutically, cosmetically or edibly acceptable salt thereof, or a mixture thereof, together with a pharmaceutically, cosmetically or edibly acceptable carrier.

According to the invention, the Cordylutene may be prepared using standard or commonly used methods. The Cordylutenes may be isolated from Cordyceps militaris. In one example of the invention, the fruiting bodies or mycelia of Cordyceps militaris were first dried to a moisture content below 10%, and the dried material then underwent a stepwise chemical treatment consisting of acid and base pre-conditioning followed by ethanol extraction under alkaline and thermal conditions.

In addition, the Cordylutenes may be synthesized through a multi-step total synthesis route. The synthesis proceeds from commercially available starting materials and involves iterative Horner-Wadsworth-Emmons (HWE) olefination, DIBAL-H reduction, and barium manganate oxidation to elongate the polyene chain and install the requisite aldehyde functionalities (see Examples 2-7). Subsequent selenium dioxide oxidation at the terminal position, followed by Jones oxidation to afford the corresponding dicarboxylic acid (see Example 8-9), enables subsequent amidation using DCC/HOBt-mediated reaction with L-valine methyl ester (see Example 10). Final hydrolysis yields Cordylutene A in analytically pure form. A summary of the synthesis of Cordylutene A is given below:

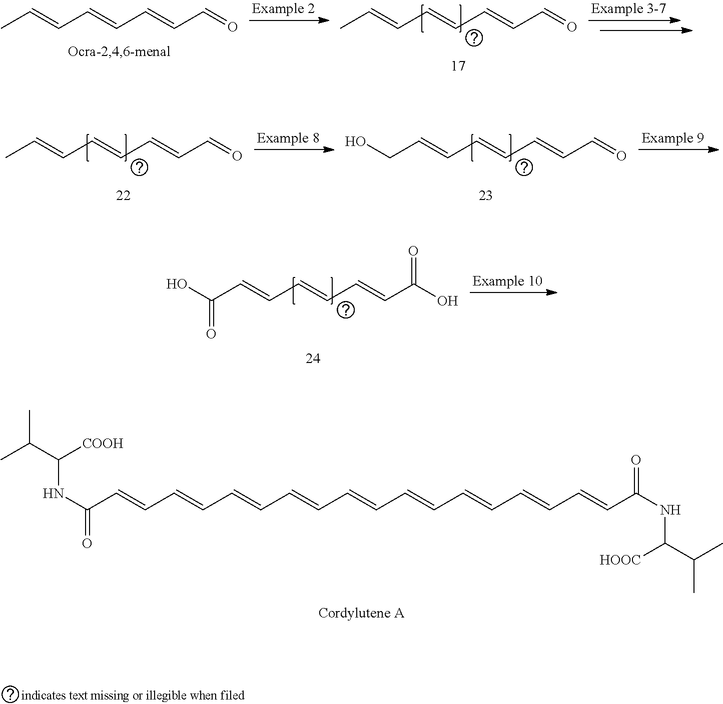

According to the invention, the structurally diverse class of the Cordylutene derivatives sharing the same core chromophore may also be prepared. As demonstrated in Examples 11 to 14, the polyene backbone may be extended or truncated to afford molecules containing different numbers of conjugated double bonds, as exemplified by shorter-chain derivatives in Examples 11 and 12, as shown below.

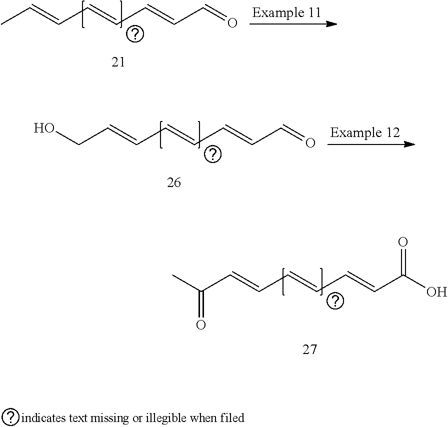

Likewise, the terminal carbonyl functionalities can be varied to include carboxylic acids, esters, amides, aldehydes, or thioesters, as illustrated in Examples 9, 10, 13, and 14, as shown below.

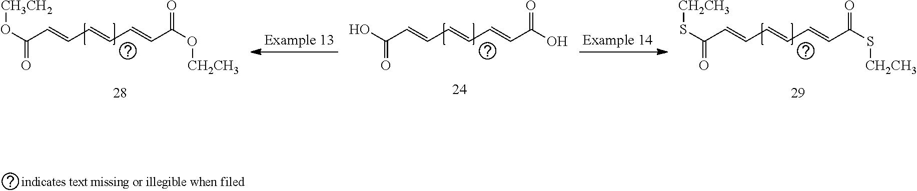

Importantly, despite variations at the terminal positions, all the compounds preserve a fully conjugated polyene system. This conserved chromophore is responsible for the distinctive blue light absorption profile exhibited by all derivatives, with UV-Vis consistently falling within the 400-500 nm range. Accordingly, the present invention broadly covers the compounds sharing the same polyene backbone but bearing different chain lengths and carbonyl functionalities, without compromising the photophysical properties central to their biological and technological applications.

As used herein, the term “subject” refers to a human or an animal, including a human or an animal. In the present invention, the subject or patient is a human with an ocular disease, or a group in need thereof for prevention or treatment of an ocular disease, such as dry eye disease, cataracts, glaucoma, and macular degeneration or age-related macular degeneration (AMD).

As used herein, the term “pharmaceutically, cosmetically or edibly acceptable salt” includes acid addition salt. The “pharmaceutically, cosmetically or edibly acceptable salt” refers to those salt which retain the biological effectiveness and properties of the free base, which are formed with inorganic acid such as hydrochloric acid, hydrobromic acid, sulfuric acid, nitric acid, phosphoric acid, and the like, and organic acids such as acetic acid, propionic acid, pyruvic acid, maleic acid, malonic acid, succinic acid, fumaric acid, tartaric aid, citric acid, benzoic acid, mandelic acid, methanesulfonic acid, ethanesulfonic acid, p-toluenesulfonic acid, salicylic acid, trifluoroacetic acid and the like.

According to the invention, the “pharmaceutical, cosmetical or edible composition composition” may be formulated with a pharmaceutically, cosmetically or edibly acceptable carrier by standard or commonly used methods. One example of the pharmaceutically, cosmetically or edibly acceptable carrier is hydrogel or water.

The term “a pharmaceutically, cosmetically, or edibly acceptable carrier” used herein refers to a carrier(s), diluent(s) or excipient(s) that is acceptable, in the sense of being compatible with the other ingredients of the formulation and not deleterious to the subject to be administered with the pharmaceutical, cosmetical, or edible composition. Any carrier, diluent or excipient commonly known or used in the field may be used in the invention, depending to the requirements of the pharmaceutical, cosmetical, or edible formulation. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents.

According to the invention, the Cordylutene has an exceptional blue light absorption capability. By a spectroscopic analysis, it has been demonstrated that the Cordylutene exhibited significant UV absorption in the blue light spectrum (400-500 nm); wherein the absorption peaks in the range were primarily attributed to the all-trans polyene chains, in which the core structure of the Cordylutene contains double bonds in the conjugated π-electron system, and shifts the absorption maximum about 30 nm in the same direction, resulting in bathochromic and hyperchromic shifted in absorption. This continuous carbon-carbon double bond (n=7-11) of the core structure is responsible for the effective absorption of blue light (400-500 nm), ensuring that the Cordylutene mitigates the penetration of harmful blue light into the eye. Importantly, this blue light absorption capability of the Cordylutene remains consistent regardless of the substituents attached. The presence of various substituents at both ends of the core structure does not significantly affect the capability of absorbing blue light. This robustness in blue light absorption is a key benefit, as it ensures that the Cordylutenes provide ocular protection across different molecular variants.

According to the invention, the Cordylutene provides an effect in inhibiting the oxidative stress induced by blue light exposure. Analysis using the 2′,7′-dichlorofluorescin diacetate (DCFDA) intracellular oxidative stress detection kit and fluorescence staining showed that the levels of intracellular ROS and RNS in the Cordylutene A group sharply decreased by 33.0% compared to the light-induced damage negative control group.

According to the invention, the Cordylutene exhibits superior antioxidant capacity comparing to lutein by assessing the antioxidant activity via 2,2-Diphenyl-1-picrylhydrazyl (DPPH) radical scavenging.

Another significant advantage of the Cordylutenes according to the present invention is the higher water solubility. As compared to other eye-protective substances such as lutein or zeaxanthin, which is non- or less water-soluble, the Cordylutenes exhibit higher polarity. This higher polarity leads to a better water solubility, which offers several benefits for bio-absorption, and bio-availability. In the examples of the present invention, the compounds having the Structures (11) and (13) particularly exhibited significantly higher polarity due to the presence of multiple hydroxyl groups at the both ends of the core structure. This high polarity results in excellent solubility in ethanol aqueous solution. Empirical evidence from the solubility tests of the Cordylutenes support their higher solubility in water and ethanol mixtures as compared to solvents such as acetone, ether, and n-hexane. This solubility profile showed that Cordylutenes are highly polar compounds.

The Cordylutenes offer a superior profile for ocular protection due to their robust blue light absorption capabilities and better water solubility. These Cordylutenes having such advantages are potential to develop a highly effective agent/medicament/food supplement with benefits over the existing lipid-soluble alternatives such as lutein, for the prevention and/or treatment of blue light-induced ocular diseases, selected from the group consisting of dry eye disease, cataracts, glaucoma, and macular degeneration or AMD.

The present invention is further illustrated by the following examples, which are provided for the purpose of demonstration rather than limitation.

### EXAMPLES

### Example 1. Preparation and Identification of Cordylutenes A and B

### Materials and Method

### Preparation and Purification of Cordylutenes

The fruiting bodies or mycelia of Cordyceps militaris were first dried to a moisture content below 10%. The dried material then underwent a stepwise chemical treatment consisting of acid and base pre-conditioning followed by ethanol extraction under alkaline and thermal conditions.

Specifically, the dried material was immersed in a 0.1 M trifluoroacetic acid (TFA) aqueous solution and subjected to reciprocal shaking for 2 hours. After filtration, the solid residue was treated with 0.05 M sodium bicarbonate solution for 1 hour. The neutralized residue was then extracted using 70% ethanol. During this extraction, sodium hydroxide was added to adjust the pH to 10-12, and the mixture was heated at 60° C. for 2 hours.

Following the alkaline treatment, the mixture was filtered to separate the ethanol extract from the residue. The filtrate was then acidified to pH 2-3 and refrigerated below 10° C. for 16-20 hours. Orange-red precipitates formed during this step, which were collected by filtration and freeze-dried to remove residual acid. The resulting material, which was enriched in Cordylutene analogs, was subjected to further structural analysis.

The Cordylutenes contained similar structures, which were further analyzed using High-Performance Liquid Chromatography (HPLC). The analysis conditions included a mobile phase of 50% acetonitrile (ACN) with 0.1% formic acid, a stationary phase of a C₁₈ column (250×4.6 mm), and a photodiode array detector (PDA) for detection.

The Cordylutenes were subjected to column chromatography using silica gel as the stationary phase and a water/methanol mixture as the mobile phase, which was separated in several fractions. Purification of individual compounds was achieved using a semi-preparative column with a mobile phase of 35-55% acetonitrile with either 0.1% formic acid or 0.1% ammonium acetate and a C₁₈ column (250×10 mm). In the following examples, the new 16 compounds were identified and labeled as the compounds of Structure (1) to Structure (16).

### Identification of General Structure

The structural elucidation of the compounds was performed using infrared spectroscopy (IR), high-field nuclear magnetic resonance (NMR) spectroscopy, and high-resolution tandem mass spectrometry (HIRMS/MS). Each compound's structure was determined based on the spectral data obtained from these analytical techniques.

### Chiral Analysis of Acid Hydrolysates

The chirality of each amino acid in the compounds of Structures (1) and (2) was analyzed by HPLC. Each compound (1 mg) was hydrolyzed with 6M hydrochloric acid (0.5 mL) in a sealed tube at 110° C. for 20 hours. After cooling, the hydrolysate was processed through a Sep-Pak C₁₈ SPE column (0.5 g, Waters) to remove the acid. The analyte, eluted with aqueous methanol, was then subjected to chiral analysis on a Chirex 3126 (D)-penicillamine column. The eluent system contained 0.6 mM copper sulfate, and the flow rate was maintained at 1.0 mL/min in isocratic mode. The detector was set to monitor at UV 260 nm.

### LC-HRMS/MS Analysis

The analysis was carried out using Ultraperformance Liquid Chromatography (UPLC) with a gradient starting at 35% acetonitrile with 2 mM ammonium formate and 0.1% (v/v) formic acid, and increasing to 68% over 12 minutes at a flow rate of 0.3 mL/min. The column oven temperature was set to 40.0° C., and the PDA settings included a minimum and maximum wavelength of 200 nm and 800 nm, respectively. Samples were then analyzed using Thermo Scientific™ Q Exactive Focus Orbitrap HRMS with HESI as the ion source. The spray voltage was set to 3500V for positive ionization mode and 2500V for negative ionization mode, with capillary and probe heater temperatures at 256° C. and 412.5° C., respectively. Polarity was set to positive. Resolutions were 70000 for MS¹ and 17500 for MS². Full mass scan parameters included an AGC target of 1e6. MS¹ spectra were acquired in profile mode, while MS² spectra were acquired using an isolation window of 3.0 m/z with stepped NCE settings of 10, 20, and 40.

### In Silico Analysis

The quantification table and MS² spectral summaries were uploaded to the GNPS network platform for molecular networking analysis using the FBMN workflow. The cytoscape was used to visualize the molecular network.

### Results

### Cordylutene A: Compound of Structure (1)

The Compound of Structure (1), isolated as an orange solid and named as Cordylutene A, was determined to have a molecular formula of C₃₀H₃₈N₂O₆, as evidenced by its ¹³C NMR spectrum and HRESIMS analysis.

The IR spectrum revealed key absorptions at 3500-3300 cm⁻¹, indicating the presence of hydroxy groups, and at 3268 cm⁻¹, which suggested the presence of NH groups. The absorption at 1712 cm⁻¹ corresponded to a carbonyl group (C═O), while the bands at 1634 and 1527 cm⁻¹ were indicative of C═C stretching vibrations. An additional band at 1000 cm⁻¹ suggested the presence of trans form C≡C bending.

The ¹H NMR data (DMSO-d₆, 800 MHz) provided further insight into the structure. The spectrum showed four three-proton doublets at δ_{H} 0.87 (12H, d, J=6.0 Hz, H-4′, 5′, 4″, 5″), indicating the presence of methyl groups. The multiplet at δ_{H} 2.06 (2H, m, H-3′, 3″) suggested methine protons. A triplet at δ_{H} 4.18 (2H, t, J=6.6 Hz, H-2′, 2″) was assigned to other set of heteroatom-substituted methine protons. The series of peaks between δ_{H} 6.24 and 7.08 corresponded to olefinic protons, indicating the presence of multiple double bonds. A singlet at 8.05 (2H, NH) confirmed the presence of amide protons.

The ¹³C NMR spectrum (DMSO-d₆, 200 MHz) coupled with the HSQC spectrum of 1 revealed a total of 30 carbon signals. Notably, signals at δ_{C} 18.2, 19.4, and 30.1 corresponded to methyl carbons (C-5′, 5″ and C-4′, 4″), while δ_{C} 57.8 (C-2′, 2″) indicated methine carbons. The downfield shifts at δ_{C} 124.9, 131.1, 133.2, 133.8, 134.4, 135.1, 136.2, 139.0, and 139.3 were indicative of sp²-hybridized carbons in double bonds. The carbonyl carbons were identified by their distinctive downfield signals at δ_{C} 165.3 (C-1, 20) and δC 173.3 (C-1′, 1″).

The key COSY spectrum displayed two sets of mutual-coupled protons of NH/H-2′ and NH/H-2″, which were further supported by key cross-peaks of NH/C-2′ and NH/C-2″ in the H2BC spectrum. HMBC spectrum manifests the correlations from H-5′ and H-5″ to C-2′ and H-2″, C-3′ and H-3″, C-4′ and H-4″; from H-4′ and H-4″ to C-2′ and H-2″; from H-2′ and H-2″ to C-1′ and H-1″; from H-2 and H-19 to C-1 and C-20, C-4 and C-17; from H-5 and H-16 to C-3 and C-18, C-7 and C-14; from C-3 and C-18 to C-1 and C-20.

Accordingly, the structure of Cordylutene A was determined as shown below:

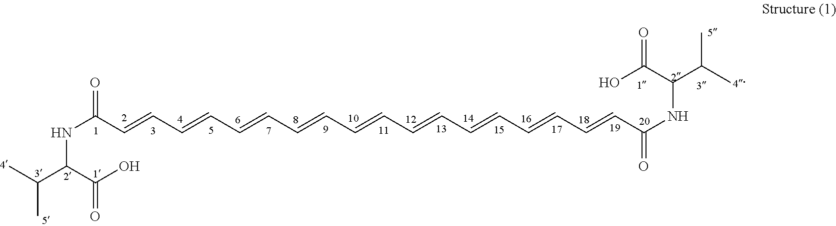 [SMILES: CC(C)C(NC(=O)/C=C/C=C/C=C/C=C/C=C/C=C/C=C/C=C/C=C/C(=O)NC(C(=O)O)C(C)C)C(=O)O]

The Cordylutene A was identified to have valine groups attached at both ends of its structure, as determined through structural elucidation. To verify the stereo-chemistry of the amino acids at both termini, acid hydrolysis was performed followed by HPLC chiral analysis of the resulting hydrolysate. The chiral analysis revealed that the valine residues were in the L-configuration. This finding further ensured the correct identification and stereochemical configuration of Cordylutene A, validating the accuracy of the initial structural determination.

### Cordylutene B: Compound of Structure (2)

The Compound of Structure (2), isolated as an orange solid and named as Cordylutene B, was determined to have a molecular formula of C₃₁H₄₀N₂O₆, as evidenced by its ¹³C NMR spectrum and HRESIMS analysis.

The ¹H NMR data (CD₃OD, 800 MHz) provided further insight into the structure. The spectrum showed four three-proton doublets at δ_{H} 0.92 (3H, t, J=8.0 Hz, H-5″), 0.93 (3H, d, J=6.4 Hz, H-4′), 0.94 (3H, d, J=7.2 Hz, H-6″), and 0.95 (3H, d, J=7.2 Hz, H-5′), indicating the presence of methyl groups. Additional multiplets of methylene proton was observed at δ_{H} 1.17 (1H, m, H-4″b) and 1.56 (1H, m, H-4″a), along with methine protons at δ_{H} 1.91 (1H, m, H-3″) and 2.20 (1H, m, H-3′). Methine protons adjacent to an amino group appeared as doublets at δ_{H} 4.34 (1H, d, J=4.8 Hz, H-2′) and 4.36 (1H, d, J=4.8 Hz, H-2″). The spectrum also displayed a series of peaks corresponding to olefinic protons, with signals at δ_{H} 6.15 (2H, d, J=14.0 Hz, H-2, 19), 6.41 (2H, t, J=12.0 Hz, H-4, 17), 6.42 (8H, m, H-6, 8, 9, 10, 11, 12, 13, 15), 6.50 (2H, dd, J=14.0, 13.2 Hz, H-7, 14), 6.64 (2H, t, J=13.2 Hz, H-5, 16), and 7.18 (2H, m, H-3, 18). These peaks suggested the presence of multiple double bonds within the structure.

The ¹³C NMR spectrum (CD₃OD, 200 MHz) further clarified the structure, revealing a total of 31 carbon signals. Notably, signals at δ_{C} 12.2 (C-5″), 16.4 (C-6″), 18.3 (C-4′), and 20.2 (C-5′) corresponded to methyl carbons, while δ_{C} 26.1 (C-4″) indicated a methylene carbon. Signals at δ_{C} 32.9 (C-3′), 39.6 (C-3″), 61.1 (C-2′), and 61.5 (C-2″) indicated methine carbons. The downfield shifts at δ_{C} 125.2 (C-2, 19), 131.9 (C-4, 17), 134.0 (C-6, 15), 134.9 (C-8, 13), 135.5 (C-10, 11), 136.4 (C-9, 12), 137.7 (C-7, 14), 140.7 (C-5, 16), and 141.5 (C-3, 18) were indicative of sp²-hybridized carbons in double bonds. The carbonyl carbons were identified by their distinctive downfield signals at δ_{C} 170.3 (C-1, 20) and 178.5 (C-1′, 1″).

The NMR data of Cordylutene B were almost identical to those of Cordylutene A except for the presence of an additional methylene group at δ_{H} 1.17 (1H, m, H-4″b) and 1.56 (1H, m, H-4″a)/δ_{C} 26.1 (C-4″), which was not observed in Cordylutene A. This difference suggests that Cordylutene B has one more carbon atom in the form of an ethyl group compared to Cordylutene A. This structural modification is further supported by the ¹³C NMR, HMBC data and HRESIMS analysis.

Accordingly, the structure of Cordylutene B was determined as shown below:

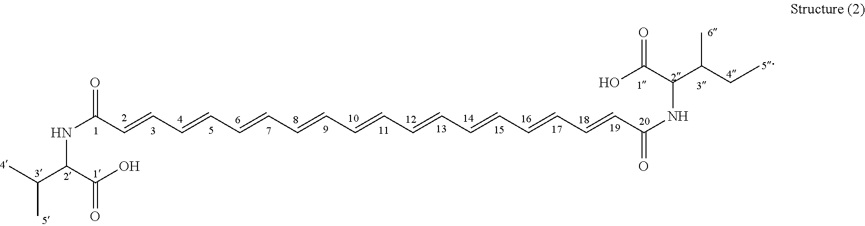 [SMILES: CCC(C)C(NC(=O)/C=C/C=C/C=C/C=C/C=C/C=C/C=C/C=C/C=C/C(=O)NC(C(=O)O)C(C)C)C(=O)O]

The Cordylutene B was identified as the ethyl analogue of Cordylutene A, highlighting the structural change with the substitution of an ethyl group at the 4″ position. Structural elucidation determined that Cordylutene B has valine and isoleucine groups attached at its termini. To verify the stereochemistry of these amino acids, acid hydrolysis followed by HPLC chiral analysis of the hydrolysate was conducted. The chiral analysis results indicated that both the valine and isoleucine residues were in the L-configuration. This confirmation reinforced the correct identification of its stereochemical configuration and validated the accuracy of the initial structural determination.

### MS² Analysis of Cordylutenes

The structural elucidation of Cordylutene A and Cordylutene B were confirmed through detailed NMR and HRESIMS analysis. As shown below, Cordylutene A was identified to have valine groups attached at both ends of the structure thereof, while Cordylutene B featured valine and isoleucine groups at the termini thereof.

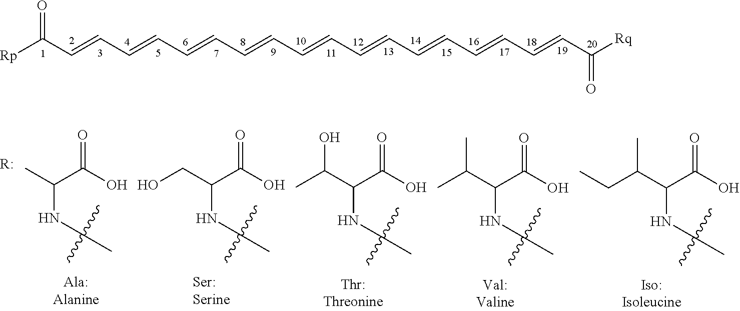

In the MS/MS analysis, both Cordylutene A and Cordylutene B exhibited three prominent signals. The signals of Cordylutene A were observed at m/z 131.0491, 406.2011, and 406.2011. The signal at m/z 131.0491 corresponds to the long carbon chain backbone (C₁₉H₁₈O) as shown below, indicating that this core structure remains intact during fragmentation:

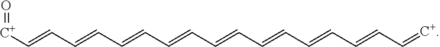

The signals at m/z 406.2011 represent the loss of one of the valine groups, showcasing major cleavage sites in the molecule. The MS/MS analysis of Cordylutene B showed signals at m/z 131.0491, 406.2010, and 420.2167. The signal at m/z 131.0491, which is similar to Cordylutene A, corresponded to the carbon chain backbone. The signal at m/z 420.2167 ([M-C₅H₁₀NO₂]⁺) results from the loss of the valine group, while the signal at m/z 406.2010 ([M-C₆H₁₃NO₂]⁺) results from the loss of the isoleucine group, showing the structural difference between the two compounds.

The fragmentation patterns observed in Cordylutene A and Cordylutene B involve the cleavage of the N—CO bond in the amide groups, leading to the formation of acylium ions. These fragments, identified as [M-Rₚ]⁺ and [M-R_{q}]⁺ (as shown below), indicate the points where the molecule splits due to the loss of each group:

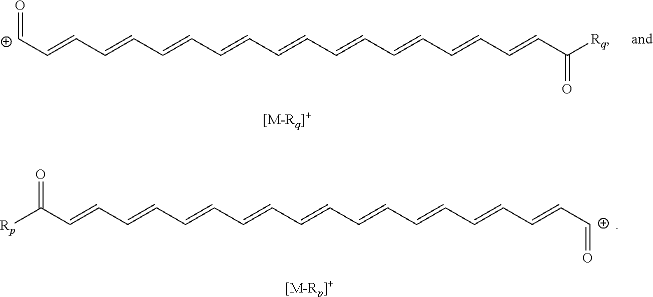

The presence of these acylium ions are particularly notable, as it indicates a stable and common cleavage pathway due to the conjugation with the α,β-unsaturated carbonyl group of the amide. This fragmentation behavior confirmed the proposed structures derived from NMR and HRESIMS data.

The MS/MS spectra prominently feature acylium ion peaks, affirming the presence of amide groups and their characteristic cleavage behavior. This comprehensive analysis validates the structures of Cordylutene A and Cordylutene B, providing critical insights into their stability and cleavage behavior. These findings support the identification and characterization of these compounds and establish a reliable framework for studying similar compounds.

### Structural Deduction of Cordylutenes

Following the detailed analysis of Cordylutene A and Cordylutene B, fourteen additional derivatives were identified using MS¹ and MS² analysis as shown in Table 2. The identification and structural elucidation of these derivatives were achieved through the careful examination of their molecular ions and the specific fragment ions resulting from the loss of various amino acid substituents.

TABLE 2
MS¹ and MS² data of the compounds of Structures (1)-(16)

| | | | | | | HRESIMS/MS m/z | | |
|---|---|---|---|---|---|---|---|---|
| | Substituent | | HRESIMS m/z | | | | | base |
| Structure | Rₚ | R_{q} | [M + H]⁺ | Formula | [M + H]⁺_{Calc.} | [M − R_{q}]⁺ | [M − Rₚ]⁺ | peak |
| (1) | Val | Val | 523.2798 | C₃₀H₃₉N₂O₆ | 523.2808 | 406.2011 | — | 131.0491 |
| (2) | Val | Ile | 537.2956 | C₃₁H₄₁N₂O₆ | 537.2965 | 406.2010 | 420.2167 | 131.0491 |
| (3) | Ala | Ala | 467.2173 | C₂₆H₃₁N₂O₆ | 467.2182 | 378.1699 | — | 131.0491 |
| (4) | Ala | Ile | 509.2647 | C₂₉H₃₇N₂O₆ | 509.2652 | 378.1694 | 420.2164 | 131.0491 |
| (5) | Ala | Ser | 483.2116 | C₂₆H₃₁N₂O₇ | 483.2131 | 378.1692 | 394.1635 | 131.0491 |
| (6) | Ala | Thr | 497.2283 | C₂₇H₃₃N₂O₇ | 497.2288 | 378.1697 | 408.1803 | 131.0491 |
| (7) | Ala | Val | 495.2490 | C₂₈H₃₅N₂O₆ | 495.2495 | 378.1688 | 406.2008 | 131.0491 |
| (8) | Ile | Ile | 551.3118 | C₃₂H₄₃N₂O₆ | 551.3121 | 420.2164 | — | 131.0491 |
| (9) | Ile | Ser | 525.2587 | C₂₉H₃₇N₂O₇ | 525.2601 | 420.2165 | 394.1654 | 131.0491 |
| (10) | Ile | Thr | 539.2767 | C₃₀H₃₉N₂O₇ | 539.2757 | 420.2163 | 408.1801 | 131.0491 |
| (11) | Ser | Thr | 513.2216 | C₂₇H₃₃N₂O₈ | 513.2237 | 394.1648 | 408.1802 | 131.0491 |
| (12) | Ser | Val | 511.2422 | C₂₈H₃₅N₂O₇ | 511.2444 | 394.1646 | 406.2011 | 131.0491 |
| (13) | Thr | Thr | 527.2380 | C₂₈H₃₅N₂O₈ | 527.2393 | 408.1802 | — | 131.0491 |
| (14) | Thr | Val | 525.2598 | C₂₉H₃₇N₂O₇ | 525.2601 | 408.1801 | 406.2009 | 131.0491 |
| (15) | Val | OH | 424.2114 | C₂₅H₃₀NO₅ | 424.5078 | 406.2010 | — | 131.0491 |
| (16) | Thr | OH | 426.1906 | C₂₄H₂₈NO₆ | 426.4804 | 408.1802 | — | 131.0491 |

The presence of valine was confirmed by the fragment ion at m/z 406.2010. This fragment represents the core structure plus valine after the loss of the other substituent, demonstrating that any compound exhibiting this fragment ion contains at least one valine group. This result showed that all of the compounds of Structures (1), (2), (7), (12), (14), and (15) contained valine, as evidenced by the presence of this fragment ion in their MS² spectra.

The presence of the fragment ion was confirmed at m/z 420.2167, which represented the core structure plus isoleucine after the loss of the other substituent, demonstrating that any compound exhibiting this fragment ion contains at least one isoleucine group. This result showed that all of the compounds of Structures (2), (4), (8), (9), and (10) contained isoleucine, as evidenced by the presence of this fragment ion in their MS² spectra.

The presence of threonine was confirmed by the fragment ion at m/z 408.1802. This fragment represents the core structure plus threonine after the loss of the other substituent, demonstrating that any compound exhibiting this fragment ion contained at least one threonine. This result showed that all the compounds of Structures (6), (10), (11), (13), (14) and (16) contained threonine, as evidenced by the presence of this fragment ion in their MS² spectra. As shown in Table 2, the compound of Structure (14) had a molecular ion [M+H]⁺ at m/z 525.2598, which was 2 Da higher than the compound of Structure (1).

The presence of serine was confirmed by the fragment ion at m/z 394.1646. This fragment represents the core structure plus serine after the loss of the other substituent, demonstrating that any compound exhibiting this fragment ion contains at least one serine. This result showed that the all compounds of Structures (5), (9), (11), and (12) contained serine, as evidenced by the presence of this fragment ion in their MS² spectra.

The presence of alanine was confirmed by the fragment ion at m/z 378.1699. This fragment represents the core structure plus alanine after the loss of the other substituent, demonstrating that any compound exhibiting this fragment ion contains at least one alanine. This result showed that the all compounds of Structures (3), (4), (5), (6), and (7) contained alanine, as evidenced by the presence of this fragment ion in their MS² spectra.

According to the results, the compound of Structure (3) had a molecular ion [M+H]⁺ at m/z 467.2173 and the fragment ion [M-Rₚ]⁺ at m/z 378.1699, confirming the presence of alanine substituents. The compound of Structure (4) had a molecular ion [M+H]⁺ at m/z 509.2647 and fragment ions at m/z 378.1694 and 420.2164, confirming alanine and isoleucine substituents. The compound of Structure (5) had a molecular ion [M+H]⁺ at m/z 483.2116 and the fragment ions at m/z 378.1692 and 394.1635, confirming the presence of alanine and serine substituents. The compound of Structure (6) had a molecular ion [M+H]⁺ at m/z 497.2283 and fragment ions at m/z 378.1697 and 408.1803, confirming the presence of alanine and threonine. The compound of Structure (8) had a molecular ion [M+H]⁺ at m/z 551.3118 and fragment ion atm/z 420.2164, confirming the presence of isoleucine substituents. The compound of Structure (9) had a molecular ion [M+H]⁺ at m/z 525.2587 and fragment ions at m/z 420.2165 and 394.1654, confirming the presence of isoleucine and serine substituents. The compound of Structure (10) had a molecular ion [M+H]⁺ at m/z 539.2767 and fragment ions at m/z 420.2163 and 408.1801, confirming the presence of isoleucine and threonine. The compound of Structure (11) had a molecular ion [M+H]⁺ at m/z 513.2216 and fragment ions at m/z 394.1648 and 408.1802, confirming the presence of serine and threonine substituents. The compound of Structure (13) had a molecular ion [M+H]⁺ at m/z 527.2380 and fragment ions at m/z 408.1802, confirming the presence of threonine substituents. The compound of Structure (14) had a molecular ion [M+H]⁺ at m/z 525.2598 and fragment ions at m/z 406.2009 and 408.1801, confirming the presence of valine and threonine substituents. The compound of Structure (15) had a molecular ion [M+H]⁺ at m/z 424.2114 and fragment ions at m/z 406.2010, confirming the presence of valine substituent. The compound of Structure (16) had a molecular ion [M+H]⁺ at m/z 426.1906 and fragment ions at m/z 408.1802, confirming the presence of threonine substituent.

In conclusion, the MS¹ and MS² data were used for identifying and structurally elucidating the 14 compounds of Structures (3) to (16), based on the structures of Cordylutene A and Cordylutene B, which are Structures (1) and (2). The consistent patterns of fragment ions, combined with the molecular ions, provided a robust framework for determining the substituents and confirming the structures of the Cordylutenes.

### Example 2: Synthesis of Deca-2,4,6,8-Tetraenal of Structure (17)

Sodium hydride (60% dispersion in oil, 0.42 g, 10.5 mmol) was added to 80 mL THF at 0° C. Triethyl phosphonoacetate (3.60 g, 16.0 mmol) was added dropwise and stirred for 1 hour. Octa-2,4,6-trienal (1.78 g, 14.5 mmol) was then added slowly, and the reaction mixture was stirred at 0° C. for 5 minutes before allowing warm to room temperature and stirring for an additional 1 hour. The reaction was quenched with 100 mL of saturated aqueous ammonium chloride and extracted with ethyl acetate (3×120 mL). The combined organic layers were washed with brine, dried with magnesium sulfate, and evaporated. The crude residue was purified by silica gel column chromatography (n-hexane:EtOAc=20:1) to afford the intermediate tetraene ester (2.30 g, 82%).

The resulting ester (2.30 g, 11.9 mmol) was dissolved in 100 mL of toluene and cooled to −80° C. DIBAL-H (1.5 M in toluene, 45.0 mmol) was added dropwise. The mixture was stirred for 15 minutes at −80° C., and then quenched with 5 mL of methanol, followed by addition of 15 mL of water. The solution was extracted with ethyl acetate, washed with brine, dried with magnesium sulfate, and evaporated to obtain the intermediate allylic alcohol. The alcohol was dissolved in dichloromethane, and barium manganate (26.90 g, 105.0 mmol) was added in portions. The suspension was stirred at room temperature for 3 hours. After completion, the mixture was filtered then evaporated. The residue was purified by silica gel column (n-hexane:EtOAc=20:1) to yield deca-2,4,6,8-tetraenal (17) (1.50 g, 70%); UV-Vis (chloroform) λₘₐₓ=348 nm; ¹H NMR (CDCl₃, 300 MHz): δ_{H} 2.07 (3H, d, J=6.0 Hz), 5.95-7.05 (7H, m), 7.36 (1H, dd, J=15.1, 10.2) and 9.60 (1H, d, J=9.2); m/z 149.09 [M+H]⁺.

### Example 3: Synthesis of Dodeca-2,4,6,8,10-Pentaenal of Structure (18)

Deca-2,4,6,8-tetraenal (17) was subjected to the same three-step elongation strategy as described in Example 2. Sodium hydride (60% in oil, 0.42 g, 10.5 mmol) and triethyl phosphonoacetate (2.49 g, 11.1 mmol) were added to THF, and stirred at 0° C. for 1 hour. Deca-2,4,6,8-tetraenal (1.50 g, 10.1 mmol) was then added, the reaction was stirred for 5 min, then allowed to warm to room temperature for 1 hour. Work-up was carried out by quenching with ammonium chloride solution, followed by extraction with ethyl acetate, dried with magnesium sulfate, and evaporated to obtain the intermediate ester (1.88 g, 85%).

The resulting ester was dissolved in toluene and cooled to −80° C. DIBAL-H (30 mmol, 1.5 M in toluene) was added dropwise. After 15 minutes, methanol and water were using to quench. The organic layer was worked up as previously described to yield the allylic alcohol, which was oxidized with barium manganate (19.48 g, 76.0 mmol) in dichloromethane for 3 hours. After filtration and chromatography (n-hexane:EtOAc=20:1), dodeca-2,4,6,8,10-pentaenal of Structure (18) was obtained (1.32 g, 75%); UV-Vis (chloroform) λₘₐₓ=371 nm; ¹H NMR (CDCl₃, 300 MHz): δ_{H} 2.06 (3H, d, J=5.9 Hz), 5.92-7.08 (9H, m), 7.40 (1H, dd, J=15.3, 10.6) and 9.61 (1H, d, J=8.8); m/z 175.11 [M+H]⁺.

### Example 4: Synthesis of Tetradeca-2,4,6,8,10,12-Hexaenal of Structure (19)

Dodeca-2,4,6,8,10-pentaenal of Structure (18) (1.32 g, 7.6 mmol) was dissolved in THE and reacted with sodium hydride and triethyl phosphonoacetate as described in Example 3. The resulting ester was reduced with DIBAL-H in toluene at −80° C., quenched with methanol and water, and then oxidized using barium manganate in dichloromethane. Purification by silica gel (n-hexane:EtOAc=20:1), afforded tetradeca-2,4,6,8,10,12-hexaenal of Structure (19) (1.00 g, 66%); UV-Vis (chloroform) λₘₐₓ=395 nm; ¹H NMR (CDCl₃, 300 MHz): δ_{H} 2.06 (3H, d, J=5.8 Hz), 5.81-7.15 (11H, m), 7.41 (1H, dd, J=15.5, 10.7) and 9.60 (1H, d, J=9.2); m/z 201.12 [M+H]⁺.

### Example 5: Synthesis of Hexadeca-2,4,6,8,10,12,14-Heptaenal of Structure (20)

Following the same procedure, tetradeca-2,4,6,8,10,12-hexaenal of Structure (19) (1.00 g, 5.0 mmol) underwent Horner-Wadsworth-Emmons reaction with sodium hydride and triethyl phosphonoacetate, DIBAL-H reduction, and barium manganate oxidation. After work-up and purification with silica gel (n-hexane:EtOAc=20:1), hexadeca-2,4,6,8,10,12,14-heptaenal of Structure (20) was obtained (0.72 g, 63%); UV-Vis (chloroform) λₘₐₓ=418 nm; ¹H NMR (CDCl₃, 300 MHz): δ_{H} 2.05 (3H, d, J=5.9 Hz), 5.80-7.15 (13H, m), 7.40 (1H, dd, J=15.1, 10.5) and 9.60 (1H, d, J=8.9); m/z 227.14 [M+H]⁺.

### Example 6: Synthesis of Octadeca-2,4,6,8,10,12,14,16-Octaenal of Structure (21)

Hexadeca-2,4,6,8,10,12,14-heptaenal of Structure (20) (0.72 g, 3.2 mmol) was converted to octadeca-2,4,6,8,10,12,14,16-octaenal using the same three-step protocol. Elution was performed with n-hexane:EtOAc=30:1, and octadeca-2,4,6,8,10,12,14,16-octaenal of Structure (21) was obtained (0.48 g, 60%); UV-Vis (chloroform) λₘₐₓ=435 nm; ¹H NMR (CDCl₃, 300 MHz): δ_{H} 2.05 (3H, d, J=6.0 Hz), 5.82-7.18 (15H, m), 7.41 (1H, dd, J=14.8, 10.6) and 9.60 (1H, d, J=8.8); m/z 253.16 [M+H]⁺.

### Example 7: Synthesis of Icosa-2,4,6,8,10,12,14,16,18-Nonaenal of Structure (22)

Octadeca-2,4,6,8,10,12,14,16-octaenal of Structure (21) (0.48 g, 1.9 mmol) was treated with sodium hydride and triethyl phosphonoacetate in THF, followed by DIBAL-H reduction and barium manganate oxidation as previously described. After silica gel purification using n-hexane:EtOAc=30:1, the product icosa-2,4,6,8,10,12,14,16,18-nonaenal of Structure (22) was obtained (0.29 g, 55%); UV-Vis (chloroform) λₘₐₓ=458 nm; ¹H NMR (CDCl₃, 300 MHz): δ_{H} 2.05 (3H, d, J=6.0 Hz), 5.82-7.14 (17H, m), 7.40 (1H, dd, J=14.9, 10.5) and 9.60 (1H, d, J=8.9); m/z 279.17 [M+H]⁺.

### Example 8: Synthesis of 20-Hydroxyicosa-2,4,6,8,10,12,14,16,18-Nonaenal of Structure (23)

Icosa-2,4,6,8,10,12,14,16,18-nonaenal of Structure (22) (0.29 g, 1.0 mmol) was dissolved in 50 mL of 1,4-dioxane. Selenium dioxide (0.13 g, 1.2 mmol) was added, and the mixture was heated to 80° C. for 12 h. After cooling, the reaction mixture was filtered and evaporated. The residue was purified by silica gel (n-hexane:EtOAc=10:1) to give 20-hydroxyicosa-2,4,6,8,10,12,14,16,18-nonaenal (7) (0.26 g, 85%); UV-Vis (MeOH) λₘₐₓ=445 nm; ¹H NMR (CDCl₃, 300 MHz): δ_{H} 3.82 (2H, d, J=5.8 Hz), 5.02 (1H, s), 5.82-7.14 (17H, m), 7.40 (1H, dd, J=15.1, 10.3) and 9.61 (1H, d, J=9.0); m/z 295.17 [M+H]⁺.

### Example 9: Synthesis of Icosanonaenedioic Acid of Structure (24)

To a stirred solution of 20-hydroxyicosa-2,4,6,8,10,12,14,16,18-nonaenal of Structure (23) (0.26 g, 0.9 mmol) in 30 mL of acetone at 0° C., freshly prepared Jones reagent (CrO₃ in H₂SO₄, 1.2 mL) was added dropwise. The reaction was stirred at 0° C. for 1 hours. The mixture was quenched with 2 mL of isopropyl alcohol. The filtrate was then added with 100 mL of saturated aqueous sodium bicarbonate and extracted with ethyl acetate (3×150 mL). The combined organic layer was washed with brine, dried with magnesium sulfate, and evaporated. The residue was purified by silica gel (DCM:MeOH:acetate=10:2:0.1) to give icosanonaenedioic acid of Structure (24) (0.16 g, 55%); UV-Vis (MeOH) λₘₐₓ=425, 449, 478 nm; ¹H NMR (DMSO-d₆, 300 MHz): δ_{H}6.22-6.64 (16H, m), 7.20 (2H, m) and 10.60 (2H, s); m/z 325.14 [M+H]⁺.

### Example 10: Synthesis of Cordylutene A (of Structure (1))

Icosanonaenedioic acid (24) (0.16 g, 0.5 mmol) was dissolved in 20 mL of DMF. To the solution were added N,N′-dicyclohexylcarbodimide (DCC, 0.21 g, 1.0 mmol), hydroxybenzotriazole (HOBt, 0.14 g, 1.0 mmol), and L-valine methyl ester (0.13 g, 1.0 mmol). The reaction was stirred for 12 h at room temperature. The filtrate was then purified by silica gel (DCM:MeOH:acetate=10:2:0.1) to yield di-(methylvalyl)-icosanonaenamide (25) (0.21 g, 76%); UV-Vis (MeOH) λₘₐₓ=420, 447, 474 nm; ¹H NMR (DMSO-d₆, 800 MHz): δH 0.87 (12H, d, J=6.0 Hz), 2.06 (2H, m), 3.62 (δH, m), 4.18 (2H, t, J=6.6 Hz), 6.24 (2H, d, J=15.2 Hz), 6.44 (2H, t, J=14.4 Hz), 6.46 (8H, m), 6.52 (2H, t, J=12.0 Hz), 6.68 (2H, t, J=12.8 Hz), 7.08 (2H, d, J=12.8 Hz), 8.05 (2H, NH); m/z 551.31 [M+H]⁺.

Di-(methylvalyl)-icosanonaenamide was dissolved in methanol, and stirred with equal volume of aqueous lithium hydroxide (1 M) at room temperature for 6 hours to hydrolyze both methyl esters. The mixture was neutralized with dilute HCl, extracted with ethyl acetate (3×20 mL), filtered, and evaporated. The product was purified by Cis-reversed phase silica gel (MeOH:H₂O:CH₂OH=70:30:0.1) to afford Cordylutene A (0.17 g, 88%); UV-Vis (MeOH) λₘₐₓ=420, 447, 474 nm; ¹H NMR (DMSO-d₆, 800 MHz): δ_{H} 0.87 (12H, d, J=6.0 Hz), 2.06 (2H, m), 4.18 (2H, t, J=6.6 Hz), 6.24 (2H, d, J=15.2 Hz), 6.44 (2H, t, J=14.4 Hz), 6.46 (8H, m), 6.52 (2H, t, J=12.0 Hz), 6.68 (2H, t, J=12.8 Hz), 7.08 (2H, d, J=12.8 Hz), 8.05 (2H, NH); m/z 523.28 [M+H]⁺.

### Example 11: Synthesis of 18-Hydroxyoctadeca-2,4,6,8,10,12,14,16-Octaenal of Structure (26)

Octadeca-2,4,6,8,10,12,14,16-octaenal of Structure (22) (0.20 g, 0.8 mmol) was dissolved in 50 mL of 1,4-dioxane. Selenium dioxide (0.13 g, 1.2 mmol) was added, and the mixture was heated to 80° C. for 12 h. After cooling, the reaction mixture was filtered and evaporated. The residue was purified by silica gel (n-hexane:EtOAc=10:1) to give 18-hydroxyoctadeca-2,4,6,8,10,12,14,16-octaenal of Structure (26) (0.17 g, 81%); UV-Vis (MeOH) λₘₐₓ=420 nm; ¹H NMR (CDCl₃, 300 MHz): δ_{H} 3.86 (2H, d, J=5.8 Hz), 5.03 (1H, s), 5.82-7.18 (15H, m), 7.40 (1H, dd, J=15.0, 10.3) and 9.60 (1H, d, J=9.1); m/z 269.15 [M+H]⁺.

### Example 12: Synthesis of Octadeca-2,4,6,8,10,12,14,16-Octaenedioic Acid of Structure (27)

To a stirred solution of 18-hydroxyoctadeca-2,4,6,8,10,12,14,16-octaenal of Structure (26) (0.17 g, 0.65 mmol) in 30 mL of acetone at 0° C., freshly prepared Jones reagent (CrO₃ in H₂SO₄, 1.0 mL) was added dropwise. The reaction was stirred at 0° C. for 1 hours. The mixture was quenched with isopropyl alcohol. The filtrate was then added with 100 mL of saturated aqueous sodium bicarbonate and extracted with ethyl acetate (3×150 mL). The combined organic layer was washed with brine, dried with magnesium sulfate, and evaporated. The residue was purified by silica gel (DCM:MeOH:acetate=10:2:0.1) to give octadeca-2,4,6,8,10,12,14,16-octaenedioic acid of Structure (27) (0.11 g, 57%); UV-Vis (MeOH) λₘₐₓ=408, 425, 444 nm; ¹H NMR (DMSO-d₆, 300 MHz): δ_{H} 6.22-6.64 (16H, m), 7.20 (2H, m) and 10.6 (2H, s); m/z 299.12 [M+H]⁺.

### Example 13: Synthesis of Diethyl Icosanonaenedioate of Structure (28)

Icosanonaenedioic acid of Structure (24) (0.10 g, 0.3 mmol) was dissolved in 10 mL of DMF. DCC (0.08 g, 0.4 mmol) and 4-dimethylaminopyridine (DMAP, 3.6 mg, 0.03 mmol) were added to the solution. The mixture was stirred at 0° C. for 15 minutes. Dried ethanol (0.06 mL, 1.0 mmol) was then added to the reaction mixture, which was allowed to warm to room temperature and stirred for 3 hours. The filtrate was then purified by silica gel (DCM:MeOH=30:1) to yield diethyl icosanonaenedioate of Structure (28) (0.08 g, 82%); UV-Vis (chloroform) λₘₐₓ=425, 450, 476 nm; ¹H NMR (DMSO-d₆, 300 MHz): δ_{H} 1.08 (δH, t, J=7.13 Hz), 3.88 (4H, q, J=7.05 Hz), 6.25-6.66 (16H, m), 7.20 (2H, m); m/z 381.20 [M+H]⁺.

### Example 14: Synthesis of S,S-Diethyl Icosanonaenedioate of Structure (29)

Icosanonaenedioic acid of Structure (24) (0.10 g, 0.3 mmol) was dissolved in 10 mL of DMF. DCC (0.08 g, 0.4 mmol) and 4-dimethylaminopyridine (DMAP, 3.6 mg, 0.03 mmol) were added to the solution. The mixture was stirred at 0° C. for 15 minutes. Ethanethiol (0.08 mL, 1.0 mmol) was then added to the reaction mixture, which was allowed to warm to room temperature and stirred for 3 hours. The filtrate was purified by silica gel (DCM:MeOH=30:1) to yield S,S-diethyl icosanonaenedioate of Structure (29) (0.08 g, 68%); UV-Vis (chloroform) λₘₐₓ=425, 449, 476 nm; ¹H NMR (DMSO-d₆, 300 MHz): δ_{H} 1.03 (δH, t, J=7.16 Hz), 2.92 (4H, q, J=7.11 Hz), 6.22-6.66 (16H, m), 7.21 (2H, m); m/z 413.16 [M+H]⁺.

### Example 15: Biotransformation of Cordylutene Derivatives

### Materials and Methods

### Cultivation and Extraction

Cordyceps hehuanensis were cultured on a solid medium until the mycelial growth fully covered the medium surface. At this growth stage, prior to initiating the illumination phase, 10 mM L-serine was supplemented into the cultivation medium. The cultures were subsequently illuminated under controlled conditions to induce metabolite production. After the completion of cultivation, the mycelia were harvested and dried to below 10% moisture content.

The dried material was subjected to the same stepwise extraction procedure described in Example 1. Specifically, the dried material was sequentially treated with 0.1 M trifluoroacetic acid and 0.05 M sodium bicarbonate, followed by alkaline ethanol extraction at pH 10-12 and 60° C. After acidification and low-temperature precipitation, orange-red solids were collected and freeze-dried to obtain Cordylutene derivatives.

### LC-HRMS/MS Analysis

Identification of the produced analogs was performed using LC-HRMS/MS following the conditions established in Example 1. Briefly, analysis was executed via UPLC with a gradient of 35-68% acetonitrile containing 2 mM ammonium formate and 0.1% formic acid at a flow rate of 0.3 mL/min. The PDA detection wavelength ranged from 200 nm to 800 nm. Mass spectrometric analyses were conducted using a Thermo Scientific™ Q Exactive Focus Orbitrap HIRMS system.

### Results

### Structural Analysis of the Compound of Structure (30)

The LC-HRMS/MS analysis indicated significant shifts in the Cordylutene metabolite profile due to the supplemental L-serine cultivation conditions. Previously abundant valine or alanine-associated Cordylutenes were undetectable. Instead, serine-associated Cordylutene derivatives were prominently observed.

Cordylutene K of Structure (11), previously identified with serine at one terminus and threonine at the other, exhibited characteristic MS/MS fragmentation ions at m/z 394.1648 and 408.1802, corresponding to the loss of threonine and serine residues, respectively. These fragmentations confirm the presence and positions of the specific amino acids in the structure, maintaining the diagnostic backbone fragment at m/z 131.0491.

A novel serine-serine substituted Cordylutene derivative, the compound of Structure (30), was detected and structurally elucidated in significant quantities. The structure of this compound is shown below:

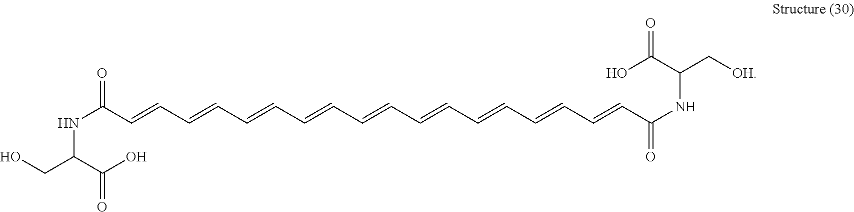 [SMILES: O=C(/C=C/C=C/C=C/C=C/C=C/C=C/C=C/C=C/C=C/C(=O)NC(CO)C(=O)O)NC(CO)C(=O)O]

The molecular ion for Structure (30) displayed diagnostic MS/MS fragmentation ions predominantly at m/z 394.1646, resulting from the cleavage and loss of serine residues from each end. This fragmentation was consistently observed and verified the presence of serine residues at each terminus, coupled with the characteristic backbone fragment at m/z 131.0491.

Additionally, under these cultivation conditions, three further novel analogs structurally related to the compounds of Structures (11) and (30) were detected. Though their precise structures remain to be elucidated, their presence was confirmed through similar fragmentation patterns observed in their MS/MS spectra.

In this example, the addition of L-serine during the late-stage cultivation of C. hehuanensis, combined with the same extraction protocol used in Example 1, led to the selective formation of serine-type Cordylutene derivatives, including the compound of Structure (11) and a newly observed bis-serine analog, the compound of Structure (30). These results indicate that the composition of Cordylutene analogs can be modulated through microbial metabolic control in combination with defined chemical processing.

### Example 16. Oxidative Stress Test for Retinal Cell Line

### Materials and Methods

### Cell Culture and Treatment

Human retinal pigment epithelial cells (ARPE-19) were cultured in a 96-well microplate at a cell density of 1.5×10⁴ cells/well. Following a 24-hour incubation period at 37° C. in a 5% CO₂ incubator to allow cell adherence, the old culture medium was removed. To induce oxidative stress, cells were pretreated with Cordylutene A (CL-A) at concentrations of 0.1 μg/mL and 1 μg/mL for 24 hours, followed by exposure to 200 μM hydrogen peroxide (H₂O₂) for 24 hours. Control groups included untreated cells and cells exposed to H₂O₂ alone.

### MTT Cell Viability Assay

Cell viability was assessed using MTT assay. After treatment, 0.05 mL of culture medium containing 1.0 mg/mL MTT was added to each well. The culture medium containing MTT reagent was removed, and 0.2 mL of DMSO was added to dissolve the formazan crystals with vigorous shaking. Absorbance values were measured using an ELISA reader at a wavelength of 570 nm, and cell viability was calculated based on the absorbance readings.

### Intracellular ROS Measurement

ROS quantification was evaluated using the in vitro ROS/RNS assay kit (DCFDA—Cellular ROS Assay Kit). The resulting supernatant from the cell lysate was mixed with 2′,7′-dichlorofluorescin diacetate (DCFDA) solution in a 96-well plate and incubated for 30 minutes at 37° C. Subsequently, the fluorescence intensity was measured using a fluorescence plate reader at 480 nm excitation and 530 nm emission.

### MDA and SOD Assay

To assess lipid peroxidation and antioxidant enzyme activity, MDA and SOD levels were determined using commercial assay kits, following the manufacturer's instructions. Cells were harvested, lysed, and centrifuged to collect supernatants.

### Results

### Cordylutene A Enhances ARPE-19 Cell Viability Under Oxidative Stress.

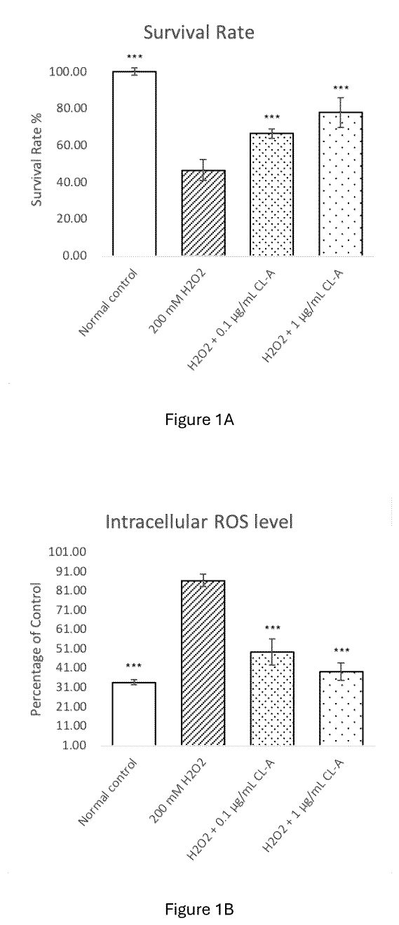

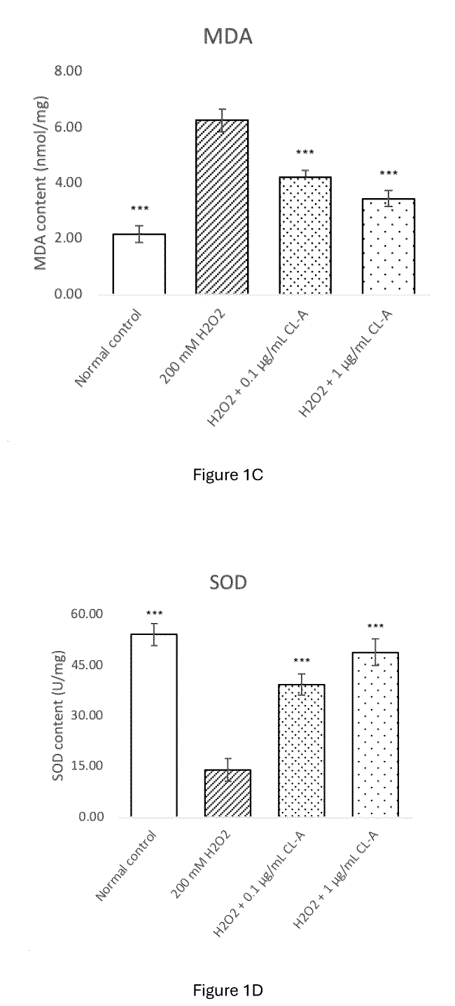

Exposure of ARPE-19 cells to 200 μM H₂O₂ for 24 h significantly reduced cell viability to 46.76% compared to the untreated control (100.48%±1.92%, p<0.001). Pretreatment with Cordylutene A at 0.1 μg/mL and 1 μg/mL effectively restored viability to 66.64% and 78.04%, respectively (p<0.001) (FIG. 1A). The effect was dose-dependent, with higher concentration showing greater protection.

### Cordylutene A Suppresses Intracellular ROS Generation.

ROS levels were markedly elevated following H₂O₂ stimulation, reaching 86.55 relative to the control baseline (33.84±1.43, p<0.001). Pretreatment with 0.1 μg/mL and 1 μg/mL Cordylutene A significantly reduced ROS accumulation to 49.63 and 39.43, respectively (p<0.001) (FIG. 1B), indicating potent antioxidative capacity.

### Cordylutene A Reduces Lipid Peroxidation.

Malondialdehyde (MDA) content, an indicator of lipid peroxidation, significantly increased in the H₂O₂ group (6.25±0.41 nmol/mg) compared to control (2.15±0.30 nmol/mg, p<0.001). Cordylutene A pretreatment dose-dependently reduced MDA levels to 4.20 nmol/mg at 0.1 μg/mL and 3.42 nmol/mg at 1 μg/mL (p<0.001) (FIG. 1C).

### Cordylutene A Restores SOD Activity in ARPE-19 Cells.

SOD enzymatic activity was significantly decreased by H₂O₂ exposure (13.97±3.46 U/mg protein) relative to control (54.09±3.31 U/mg, p<0.001). Treatment with 0.1 μg/mL and 1 μg/mL Cordylutene A markedly recovered SOD activity to 39.34 and 48.87 U/mg, respectively (p<0.001) (FIG. 1D).

Collectively, these results indicate that Cordylutene A exhibits strong protective effects against oxidative stress-induced damage in ARPE-19 cells. By improving cell viability, reducing ROS and MDA levels, and enhancing SOD activity, Cordylutene A demonstrates significant antioxidative and cytoprotective properties. These findings suggest its potential utility in developing therapeutic strategies for oxidative stress-associated retinal diseases, such as age-related macular degeneration (AMD).

### Example 17. Anti-Photoaging Test in UV-Induced Damage

### Materials and Methods

### Cell Culture and UVB Exposure

Human keratinocyte HaCaT cells were cultured in DMEM supplemented with 10% FBS and 1% penicillin-streptomycin at 37° C. in a humidified incubator containing 5% CO₂. Cells were seeded in 96-well or 6-well plates for 24 hours, then were pretreated with Cordylutene A (CL-A, 0.5 and 5 g/mL) for 24 hours. Subsequently, cells were washed with PBS, and irradiated with UVB (30 mJ/cm², 313 nm) at a distance of 5 cm. After UVB exposure, cells were incubated in medium containing Cordylutene A for 24 hours.

### MTT Cell Viability Assay

Cell viability was assessed using MTT assay. After treatment, 0.05 mL of culture medium containing 1.0 mg/mL MTT was added to each well. The culture medium containing MTT reagent was removed, and 0.2 mL of DMSO was added to dissolve the formazan crystals with vigorous shaking. Absorbance values were measured using an ELISA reader at a wavelength of 570 nm, and cell viability was calculated based on the absorbance readings.

### MDA and SOD Assay

To assess lipid peroxidation and antioxidant enzyme activity, MDA and SOD levels were determined using commercial assay kits, following the manufacturer's instructions. Cells were harvested, lysed, and centrifuged to collect supernatants.

### Results

### Cordylutene A Enhances Cell Survival After UVB Irradiation.

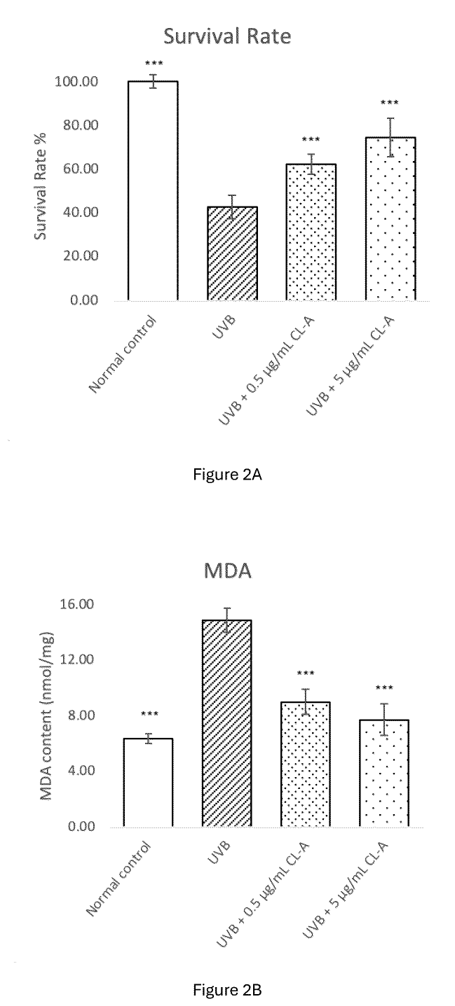

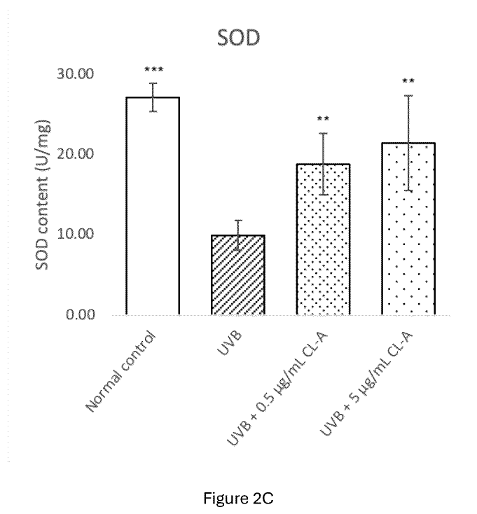

UVB exposure significantly decreased HaCaT cell viability to 42.76% compared to the non-irradiated control (p<0.001). Pretreatment with 0.5 g/mL and 5 g/mL of Cordylutene A markedly increased cell viability to 62.44% and 74.84%, respectively (p<0.001) (FIG. 2A).

### Cordylutene A Reduces UVB-Induced Lipid Peroxidation.

UVB exposure elevated MDA levels in HaCaT cells to 14.91±0.90 nmol/mg, compared with 6.35±0.36 nmol/mg in the control (p<0.001). Cordylutene A significantly suppressed MDA accumulation to 9.02 and 7.73 nmol/mg at 0.5 g/mL and 5 g/mL, respectively (p<0.001) (FIG. 2B), indicating reduced lipid peroxidation.

### Cordylutene A Enhances Antioxidant Enzyme Activity (SOD).

UVB irradiation decreased intracellular SOD activity from 27.18 U/mg (control) to 9.93 U/mg (p<0.001). Treatment with Cordylutene A restored SOD levels to 18.81 and 21.44 U/mg at 0.5 g/mL and 5 g/mL, respectively (p<0.01) (FIG. 2C), demonstrating enhancement of cellular antioxidant defense mechanisms.

Collectively, these findings demonstrate that Cordylutene A confers significant protection against UVB-induced oxidative stress in HaCaT keratinocytes. By enhancing cell survival, reducing MDA content, and restoring SOD enzyme activity, Cordylutene A displays strong antioxidant and cytoprotective properties. These results support its potential application as a natural active ingredient in anti-photoaging or therapeutic formulations targeting UV-induced cellular damage.

### Example 18. Oxidative Stress Test for Lens Cell Line

### Materials and Methods

### Cell Culture and Treatment

The human lens epithelial cell line HLE-B3 was cultured in Dulbecco's Modified Eagle's Medium (DMEM) supplemented with 10% fetal bovine serum (FBS) at 37° C. in a humidified 5% CO₂ atmosphere. For the oxidative stress model, cells were exposed to 100 μM H₂O₂ for 24 h. In the treatment groups, cells were pretreated with Cordylutene A at various concentrations (0.2, 1, 5 μM) for 2 h before H₂O₂ exposure. DMSO (0.1%) was used as vehicle control.

### MTT Cell Viability Assay

Cell viability was assessed using MTT assay. After treatment, 0.05 mL of culture medium containing 1.0 mg/mL MTT was added to each well. The culture medium containing MTT reagent was removed, and 0.2 mL of DMSO was added to dissolve the formazan crystals with vigorous shaking. Absorbance values were measured using an ELISA reader at a wavelength of 570 nm, and cell viability was calculated based on the absorbance readings.

### Intracellular ROS Measurement

ROS quantification was evaluated using the in vitro ROS/RNS assay kit (DCFDA—Cellular ROS Assay Kit). According to the manufacturer's protocol, the cells were sonicated then centrifuged at 10,000 g for 5 minutes. The resulting supernatant was mixed with 2′,7′-dichlorofluorescin diacetate (DCFDA) solution in a 96-well plate and incubated for 30 minutes at 37° C. Subsequently, the fluorescence intensity was measured using a fluorescence plate reader at 480 nm excitation and 530 nm emission.

### Results

### Cordylutene A Preserves Cell Viability Under Oxidative Stress

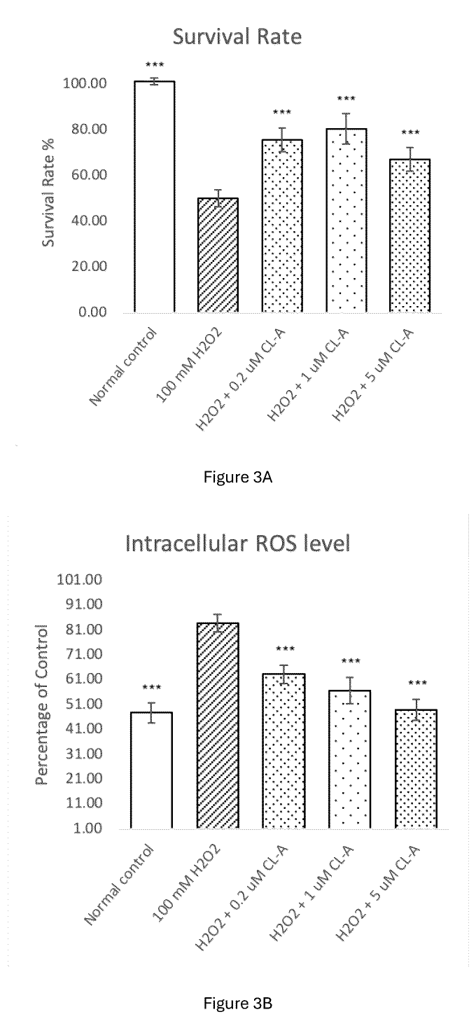

Exposure of HLE-B3 cells to 100 μM H₂O₂ for 24 hours significantly decreased cell viability to 49.88%±3.86% of the normal control (p<0.001). Pretreatment with Cordylutene A (CL-A) at 0.2 μM, 1 μM, and 5 μM markedly improved cell survival, restoring viability to 75.56%, 80.28%, and 66.88%, respectively (p<0.001 vs. H₂O₂ group). Among these, 1 μM CL-A showed the greatest cytoprotective effect (FIG. 3A), suggesting a dose-responsive, but possibly biphasic trend at higher concentrations.

Cordylutene A Reduces ROS in H₂O₂-Stimulated HLE-B3 Cells

Treatment with 100 μM H₂O₂ resulted in a significant increase in intracellular ROS levels to 83.81, compared to the untreated control group (47.73±4.06, p<0.001). Pretreatment with CL-A effectively attenuated this ROS surge in a dose-dependent manner. ROS levels were significantly reduced to 63.18, 56.65, and 48.84 in the 0.2 μM, 1 μM, and 5 μM CL-A groups, respectively (p<0.001) (FIG. 3B).

Collectively, these results demonstrate that Cordylutene A effectively protects human lens epithelial cells from oxidative stress-induced injury. It significantly restores cell viability and suppresses intracellular ROS accumulation following hydrogen peroxide exposure. Given that oxidative stress is a major contributing factor in the pathogenesis of age-related cataracts, our findings suggest that Cordylutene A has promising potential as a therapeutic or preventive agent for cataract development.

### Example 19: Hyperosmotic Stress Test for Corneal Cell Line

### Materials and Methods

### Cell Culture and Treatment

Human corneal epithelial cells (HCECs) were cultured in Dulbecco's Modified Eagle Medium/Nutrient Mixture F-12 (DMEM/F12) supplemented with 10% fetal bovine serum and 10 ng/mL human epidermal growth factor at 37° C. in a humidified atmosphere containing 5% CO₂.

To induce a dry eye disease (DED)-like environment, cells were exposed to hyperosmotic medium (500 mOsm/L), prepared by adding 90 mM sodium chloride to the control medium (312 mOsm/L). Experimental groups included normal control (312 mOsm/L), hyperosmotic model (500 mOsm/L), and hyperosmotic conditions with Cordylutene A at different concentrations. Cells were pretreated with Cordylutene A at concentrations of 0.2 μM, 1 μM, or 5 μM for 2 hr before hyperosmotic stimulation.

### RNA Extraction and Quantitative Real-Time PCR (RT-qPCR)

After 3 hours of hyperosmotic stimulation, total RNA was extracted using TRIzol reagent according to the manufacturer's instructions. Reverse transcription was performed to synthesize cDNA. Quantitative PCR was carried out, targeting human IL-1β, IL-6, TNF-α with GAPDH serving as the internal control.

### Enzyme-Linked Immunosorbent Assay (ELISA)

Conditioned media were collected after 24 hours of stimulation. The concentrations of IL-1β, IL-6, and TNF-α in the supernatants were measured using commercially available ELISA kits, following the manufacturer's protocols.

### Results

### Pro-Inflammatory Cytokine Expression in HCECs Under Hyperosmotic Stress

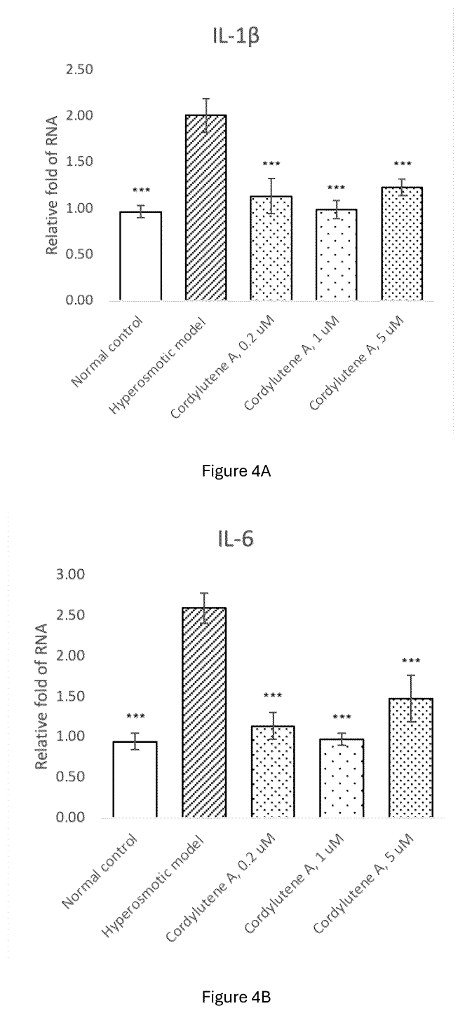

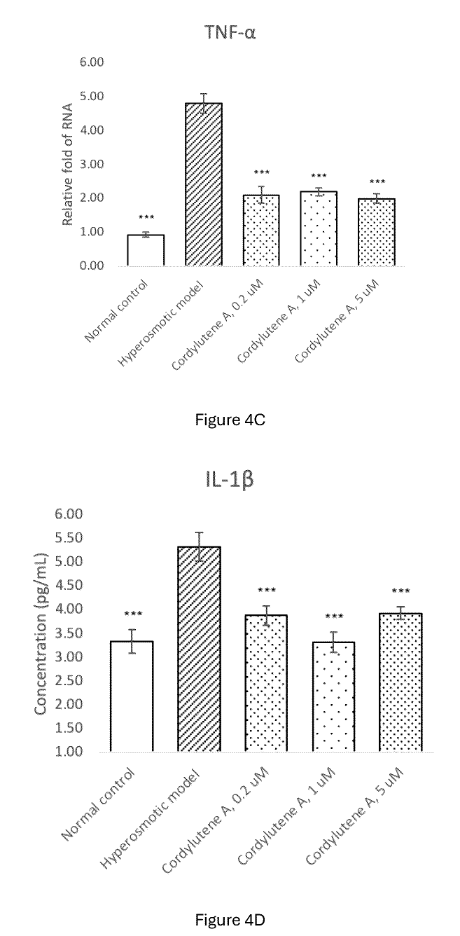

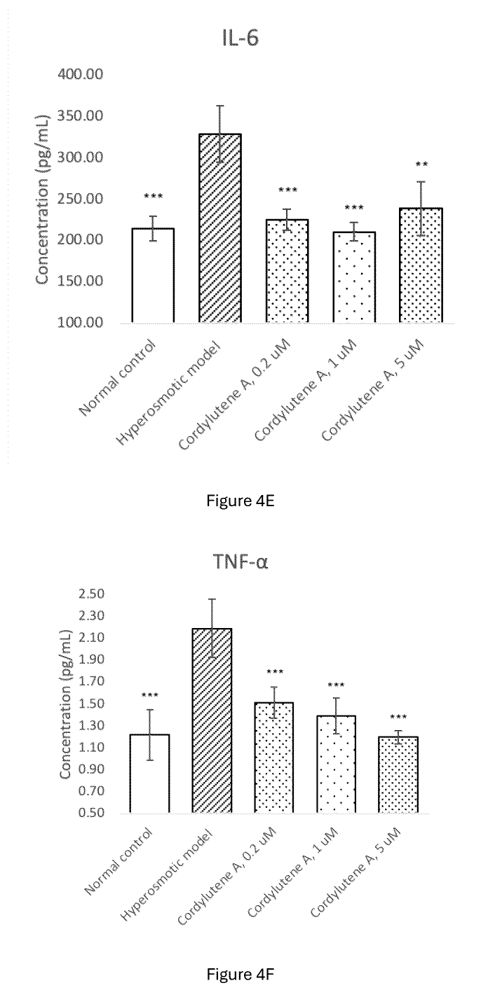

Hyperosmotic stress markedly induced the expression of pro-inflammatory cytokines in HCECs. Compared to the normal control, the mRNA levels of IL-1β and IL-6 were elevated by approximately 2-3 fold, and TNF-α increased by over 5-fold under 500 mOsm/L treatment (FIG. 13A-C). Pretreatment with Cordylutene A (CL-A) attenuated these elevations in a concentration-sensitive manner. At 0.2 μM, the expression of all three cytokines was moderately reduced (approx. 1.5-2-fold decrease compared to the hyperosmotic group), and 1 μM Cordylutene A nearly normalized IL-1β and IL-6 expression. While TNF-α remained elevated at 1 μM, treatment with 5 μM Cordylutene A led to an approximately 2.5-fold reduction in TNF-α compared to the hyperosmotic group (P<0.001; FIGS. 4A, 4B and 4C).

Protein expression measured by ELISA showed trends consistent with the mRNA results. Hyperosmotic exposure increased secreted IL-1β and IL-6 levels by roughly 1.6-fold and 1.5-fold, respectively, and TNF-α nearly doubled relative to baseline (FIGS. 4D, 4E and 4F). Cordylutene A at 0.2, 1 and 5 μM reversed these changes. Levels of IL-1β, IL-6 and TNF-α were substantially reduced toward baseline.

These findings demonstrate that Cordylutene A effectively suppresses hyperosmotic stress-induced inflammation in corneal epithelial cells at both mRNA and protein levels. The consistent downregulation of IL-1β, IL-6, and TNF-α suggests that Cordylutene A modulates key inflammatory pathways implicated in dry eye disease (DED) pathogenesis. Given the pivotal role of epithelial inflammation in DED progression, these results support the therapeutic potential of Cordylutene A as a candidate for alleviating dry eye-associated ocular surface inflammation.

### Examples 20: Light-Induced Damage Test for Retinal Cell Line

### Material and Method

### Cell Culture and Treatment

Human retinal pigment epithelial cells (ARPE-19) were cultured in a 96-well microplate at a cell density of 1.5×10⁴ cells/well. Following a 24-hour incubation period at 37° C. in a 5% CO₂ incubator to allow cell adherence, the old culture medium was removed. The cells were then treated with 0.1 mL of culture medium containing 10 μM N-retinylidene-N-retinylethanolamine (A2E) and incubated for another 24 hours. After removing the medium containing A2E, different concentrations of test samples were added, and the cells were cultured for an additional 24 hours. The microplates were exposed to blue light (420-439 nm, 50-60 W) for 9 minutes to induce light-induced damage to the ARPE-19 cells. After blue light exposure, the cells were cultured for another 24 hours. The culture medium was then removed, and 0.05 mL of culture medium containing 1.0 mg/mL MTT was added to each well. Finally, the culture medium containing the MTT reagent was removed, and 0.2 mL of DMSO was added to dissolve the formazan crystals with vigorous shaking. Absorbance values were measured using an ELISA reader at a wavelength of 570 nm, and cell viability was calculated based on the absorbance readings.

### Intracellular Oxidative Stress Analysis

The treated ARPE-19 cells were harvested for ROS quantification using the in vitro ROS/RNS assay kit (DCFDA—Cellular ROS Assay Kit). According to the manufacturer's protocol, the cells were suspended in PBS, sonicated on ice, and centrifuged at 10,000 g for 5 minutes. The resulting supernatant was mixed with 2′,7′-dichlorofluorescin diacetate (DCFDA) solution in a 96-well plate and incubated for 30 minutes at 37° C. Subsequently, the fluorescence intensity was measured using a fluorescence plate reader at 480 nm excitation and 530 nm emission.

### Data Analysis

Data were expressed as mean±standard deviation (SD). Statistical analysis was performed using SPSS statistical software with one-way ANOVA, followed by Duncan's multiple range test or Tukey's post hoc test to analyze differences between groups. A p-value less than 0.05 was considered statistically significant. In the cell viability assay, the viability=[(mean absorbance of control or test group/mean absorbance of control group)×100%]. In the oxidative stress analysis, the oxidative stress ratio (DCF-positive cells percentage)=[(mean DCF-positive cells in control or test group/total cell count)×100%].

### Results

### Protective Effect of Cordylutene Against Blue Light-Induced Damage.

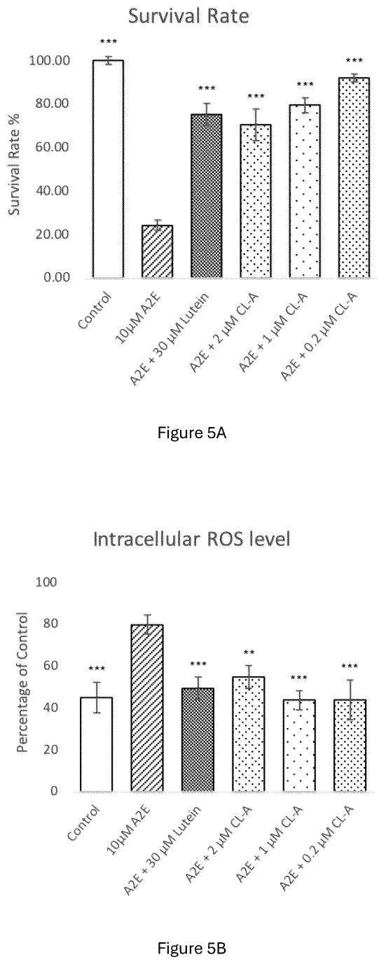

In the experimental group, 10 μM A2E was added, and cells were exposed to blue light using an LED lamp (420-439 nm) to simulate blue light-induced damage. The survival rate in the light-induced damage negative control group was 24.2%±2.3% compared to the normal control group, indicating successful establishment of the light-induced damage model. For the treatment of Cordylutene A, the survival rate of ARPE-19 at concentrations of 2, 1, and 0.2 μg/mL were 70.5%, 79.5%, and 92.1%, respectively (FIG. 5A), which were significantly higher compared to the negative control group (p<0.001). This demonstrates that Cordylutene A can help prevent blue light-induced damage to retinal cells even at low concentrations.

### Cordylutene Reduces Oxidative Stress in Blue Light-Damaged Cells.

During the cultivation of ARPE-19 cells, Cordylutene A (CL-A) was administered at concentrations of 0.2, 1, and 2 μM on days 7 and 9. On day 10, the cells were exposed to blue light irradiation to induce cellular damage. Intracellular oxidative stress levels were subsequently assessed using the DCFDA intracellular oxidative stress detection kit combined with fluorescence staining.

The results revealed a marked reduction in intracellular reactive oxygen species (ROS) levels following treatment with Cordylutene A. Specifically, cells treated with 0.2 μM Cordylutene A exhibited a 45.0% decrease in ROS levels (43.89±9.59) compared to the blue light-induced damage control group (79.77±4.51). Similarly, treatments at 1 and 2 μM Cordylutene A showed reductions of 45.3% and 31.2%, respectively (FIG. 5B).

### CONCLUSION

These findings demonstrate that the Cordylutene compounds according to the invention possess significant anti-blue light effects, enhancing cell survival rates and effectively mitigating oxidative stress induced by blue light exposure. Therefore, the Cordylutene compounds according to the invention show promising potential as protective agents against blue light-induced cellular damage and could play a role in preventing age-related macular degeneration (AMD) associated with chronic blue light exposure.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

### REFERENCE

1. Xinli Ouyang, Jing Yang, Zexin Hong, Yide Wu, Yongfang Xie, Guohui Wang, “Mechanisms of Blue Light-induced Eye Hazard and Protective Measures: A Review,” Biomedicine & Pharmacotherapy, p. 110577, 2020.
2. Deniz Goodman, Steven Ness, “The Role of Oxidative Stress in the Aging Eye,” Life, p. 837, 2023.
3. Imene Jaadane, Pierre Boulenguez, Sabine Chahory, Samuel Carré, Michèle Savoldelli, Laurent Jonet, Francine Behar-Cohen, Christophe Martinsons, Alicia Torriglia, “Retinal damage induced by commercial light emitting diodes (LEDs),” Free Radical Biology and Medicine, pp. 373-384, 2015.
4. Mohamed Abdouh, Melissa Lu, Yunxi Chen, Alicia Goyeneche, Julia Valdemarin Burnier, Miguel N Burnier Jr, “Filtering blue light mitigates the deleterious effects induced by the oxidative stress in human retinal pigment epithelial cells,” Experimental Eye Research, p. 108978, 2022.
5. Eva Chamorro, Cristina Bonnin-Arias, Maria Jesús Pérez-Carrasco, Javier Muñoz de Luna, Daniel Vázquez, Celia Sánchez-Ramos, “Effects of Light-emitting Diode Radiations on Human Retinal Pigment Epithelial Cells In Vitro,” Photochemistry and Photobiology, p. 468-473, 2013.
6. Veronika Marek, Stéphane Melik-Parsadaniantz, Thierry Villette, Fanny Montoya, Christophe Baudouin, Francoise Brignole-Baudouin, Alexandre Denoyer, “Blue light phototoxicity toward human corneal and conjunctival epithelial cells in basal and hyperosmolar conditions,” Free Radical Biology and Medicine, p. 27-40, 2018.
7. Hyo Seok Lee, Lian Cui, Ying Li, Ji Suk Choi, Joo-Hee Choi, Zhengri Li, Ga Eon Kim, Won Choi, Kyung Chul Yoon, “Influence of Light Emitting Diode-Derived Blue Light Overexposure on Mouse Ocular Surface,” PlosONE, p. e0161041, 2016.
8. Robin Haag, Nicole Sieber, Martin Heßling, “Cataract Development by Exposure to Ultraviolet and Blue Visible Light in Porcine Lenses,” Medicina, p. 535, 2021.
9. Farzin Kamari, Shahin Hallaj, Fatemeh Dorosti, Farbod Alinezhad, Negar Taleschian-Tabrizi, Fereshteh Farhadi, Hassan Aslani, “Phototoxicity of environmental radiations in human lens: revisiting the pathogenesis of UV-induced cataract,” Graefe's Archive for Clinical and Experimental Ophthalmology, p. 2065-2077, 2019.

What is claimed is:

1. A compound, named as Cordylutene, which has the structure of general Formula (I) below, or a pharmaceutically, cosmetically, or edibly acceptable salt thereof: 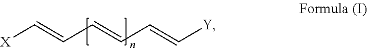
wherein
X and Y are the same or different, independently selected from the group consisting of aldehyde, ketone, carboxylic acid, ester, amide, acyl halide, acid anhydride, imide, and thioester; and
n is an integer from 5 to 9.

2. The compound of claim 1, wherein:
X is a functional group represented by Formula A: 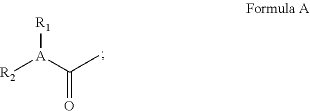
Y is a functional group represented by Formula B: 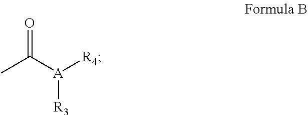
wherein:
A is a carbon, sulfur, oxygen, nitrogen, or hydrogen; and
R₁, R₂, R₃ and R₄ are the same, different or absent, independently selected from the group consisting of hydrogen, halogen, hydroxyl, cyano, nitro, thiol, alkyl, aryl, alkenyl, alkynyl, alkoxy, aryloxy, acyl, sulfonyl, amino, and heterocyclic groups.

3. The compound of claim 1, wherein:
X is a functional group represented by Formula C: 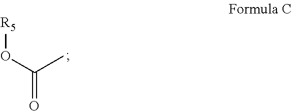
Y is a functional group represented by Formula D: 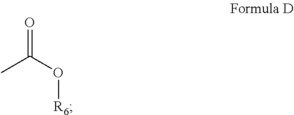
wherein
R₅ and R₆ are the same or different, independently selected from the group consisting of hydrogen, and a substituted or unsubstituted organic or inorganic radical, wherein the radical is selected from alkyl, aryl, alkenyl, alkynyl, alkoxy, aryloxy, acyl, sulfonyl, amino, heterocyclic groups, halogen, hydroxyl, cyano, or nitro.

4. The compound of claim 1, wherein:
X is a functional group represented by Formula E: 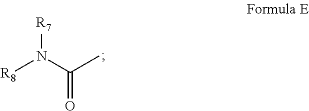
Y is a functional group represented by Formula F: 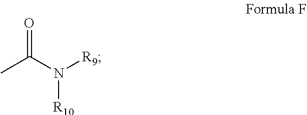
wherein
R₇ to R₁₀ are the same or different, independently selected from the group consisting of hydrogen, and a substituted or unsubstituted organic or inorganic radical, wherein the radical is selected from alkyl, aryl, alkenyl, alkynyl, alkoxy, aryloxy, acyl, sulfonyl, amino, heterocyclic groups, halogen, hydroxyl, cyano and nitro.

5. The compound of claim 1, wherein:
X is a functional group represented by Formula G: 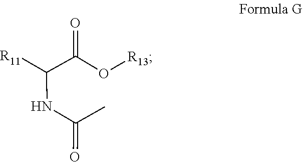
Y is a functional group represented by Formula H: 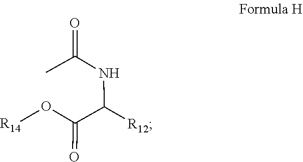
wherein
R₁₁ to R₁₄ are the same or different, independently selected from the group consisting of hydrogen, and a substituted or unsubstituted organic or inorganic radical, wherein the radical is selected from alkyl, aryl, alkenyl, alkynyl, alkoxy, aryloxy, acyl, sulfonyl, amino, heterocyclic groups, halogen, hydroxyl, cyano, or nitro.

6. The compound of claim 5, in which R₁₁ and R₁₂ are the same or different, an unsubstituted or substituted side chain derived from amino acid, selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, wherein the side chain is a substituent selected from the group consisting of methyl, isopropyl, sec-butyl, 2-butyl, thioether, benzyl, indole, hydroxymethyl, 1-hydroxyethyl, thiol, part of the cyclic structure, amide, carboxyl, long aliphatic chains, guanidinium, and imidazole.

7. The compound of claim 1, which has the structure of general Formula (I) below, or a pharmaceutically, cosmetically, or edibly acceptable salt thereof: 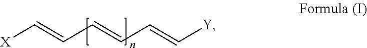
wherein
X is a functional group represented by Formula A: 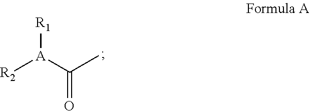
Y is a functional group represented by Formula B: 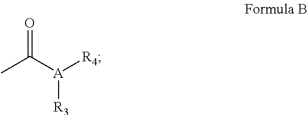
in which:
A is a sulfur, oxygen or nitrogen; and
R₁, R₂, R₃ and R₄ are the same, different or absent, independently selected from the group consisting of hydrogen, ethyl and amino acid selected from the group consisting of alanine (Ala), isoleucine (Ile), serine (Ser), threonine (Thr), and valine (Val).

8. The compound of claim 1, which is one selected from the group consisting of the compounds having the following structures

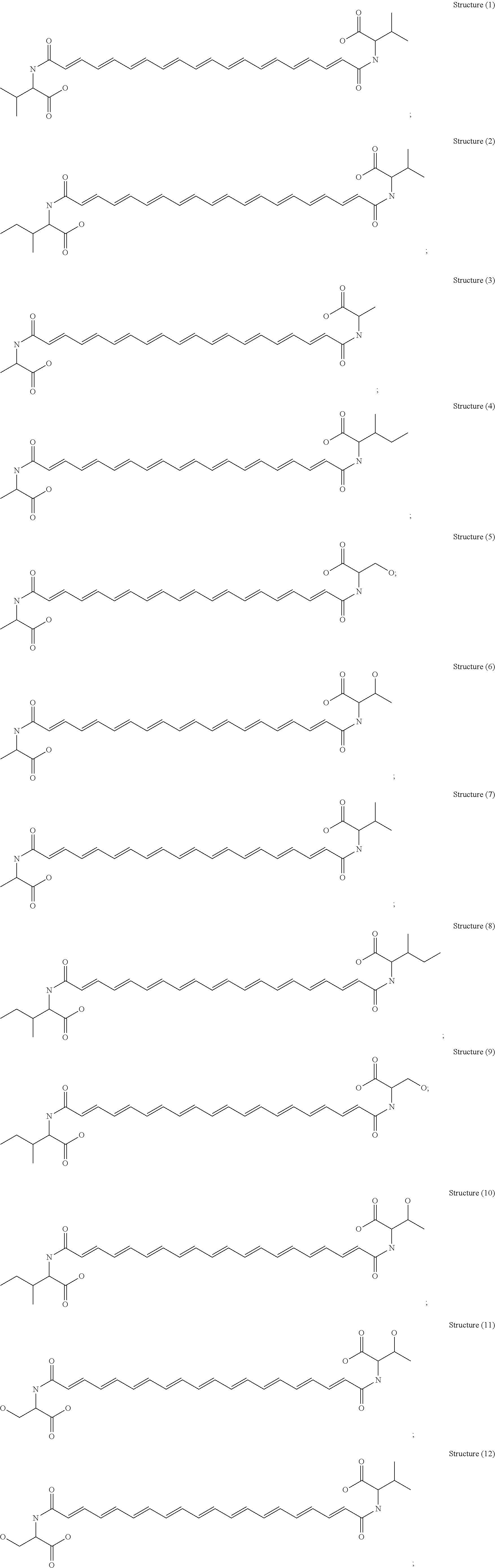

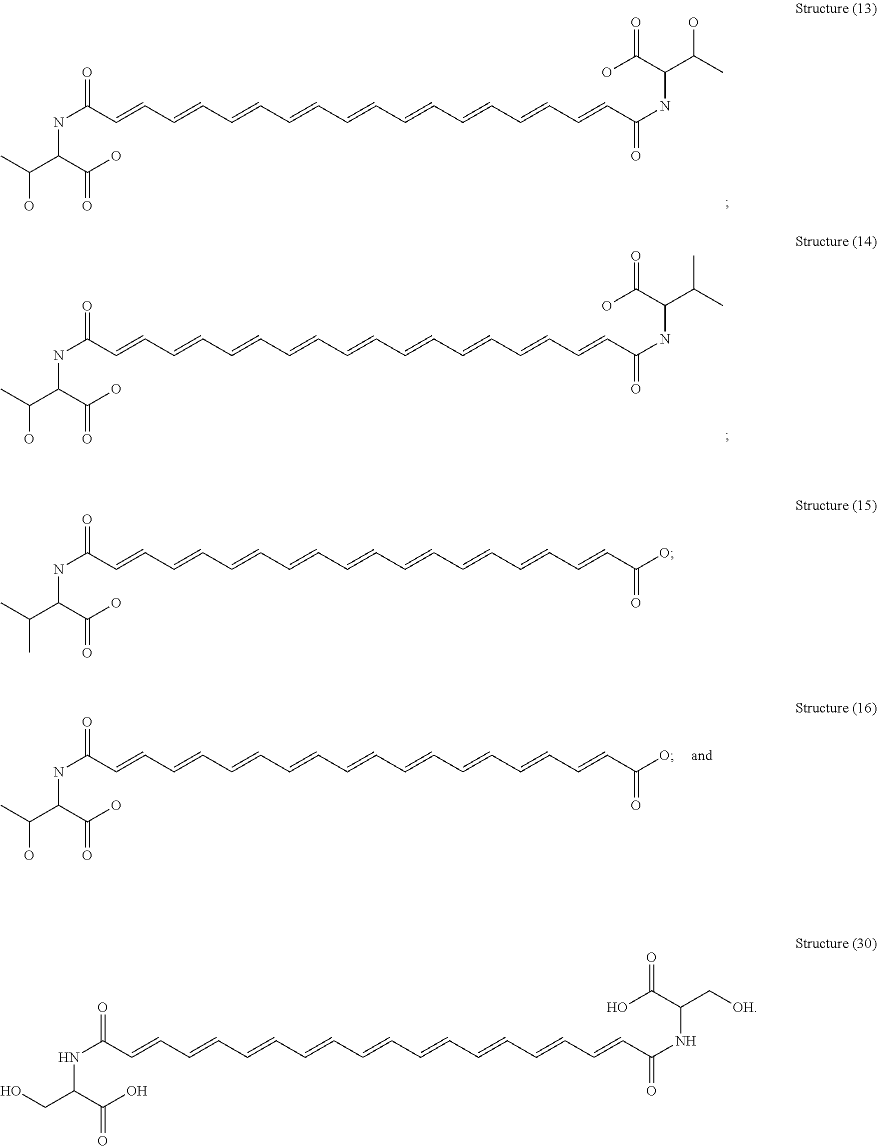

9. A method for preventing or treating an ocular disease in a subject comprising administrating the subject a pharmaceutical, cosmetical or edible composition comprising a therapeutically effective amount of the compound as defined in claim 1, or a pharmaceutically, cosmetically or edibly acceptable salt thereof, or a mixture thereof.

10. The method of claim 9, wherein the ocular disease is caused by prolonged exposure to a light source containing blue wavelengths.

11. The method of claim 9, wherein the ocular disease is selected from the group consisting of dry eye disease, cataracts, glaucoma, and macular degeneration.

12. The method of claim 9, wherein the pharmaceutical, cosmetical or edible composition is administered via oral or ophthalmic delivery.

13. A composition, which comprises a therapeutically effective amount of the compound as defined in claim 1, or a pharmaceutically, cosmetically or edibly acceptable salt thereof, or a mixture thereof, together with a pharmaceutically, cosmetically, or edibly acceptable carrier.

14. The composition of claim 13, which is used for preventing or treating an ocular disease.

15. The composition of claim 13, wherein the ocular disease is caused by prolonged exposure to a light source containing blue wavelengths.

16. The composition of claim 13, which is a pharmaceutical, cosmetical or edible composition.

17. The composition of claim 13, wherein the ocular disease is selected from the group consisting of dry eye disease, cataracts, glaucoma, and macular degeneration.
